(19) United States
(12) Patent Application Publication
(10) Pub. No.: US 2022/0254472 A1
(43) Pub. Date: Aug. 11, 2022

(54) ITERATIVE LEARNING CONTROL WITH SPARSE MEASUREMENTS FOR INSULIN INJECTIONS IN PEOPLE WITH TYPE 1 DIABETES

(71) Applicant: PRESIDENT AND FELLOWS OF HARVARD COLLEGE, Cambridge, MA (US)

(72) Inventors: Eyal DASSAU, Acton, MA (US); Marzia CESCON, Houston, TX (US); Francis J. DOYLE, III, Cambridge, MA (US)

(73) Assignee: PRESIDENT AND FELLOWS OF HARVARD COLLEGE, Cambridge, MA (US)

(21) Appl. No.: 17/625,624

(22) PCT Filed: Jul. 9, 2020
(86) PCT No.: PCT/US20/41326

§ 371 Date: Jan. 7, 2022

Related U.S. Application Data
(60) Provisional application No. 62/872,020, filed on Jul. 9, 2019.

(51) Int. Cl. G16H 20/17; A61M 5/172; A61K 38/28

(52) U.S. Cl. CPC G16H 20/17; A61M 5/1723; A61M 2205/502; A61M 2230/201; A61K 38/28

(57) ABSTRACT

The technology described herein relates to control models for artificial pancreas systems, including insulin injections in people with diabetes. The methods provided herein allow for a modular and personalized intervention for the treatment of diabetes using an iterative learning controller (ILC). The ILC allows for long-acting insulin doses to be computationally applied to track a basal glucose concentration reference, a run-to-run (R2R) control policy to update the treatment plan, that progressively meets the recommended glycemic targets.

### CROSS-REFERENCE TO RELATED APPLICATIONS

This application claims benefit under 35 U.S.C. § 119(e) of U.S. Provisional Application No. 62/872,020 filed Jul. 9, 2019, the contents of which are incorporated herein by reference in its entirety.

### TECHNICAL FIELD

The technology described herein relates to control methods for artificial pancreas systems, including insulin injections in people with diabetes.

### BACKGROUND

People with type 1 diabetes (T1D) require exogenous insulin administration for adequate blood glucose regulation. The aim of such insulin therapy is to mimic as closely as possible the physiological insulin secretion pattern in the individual without diabetes consisting in a slow basal secretion throughout the day and an augmented rate at meal times. Insulin treatment is burdensome to the patient, it entails great effort and requires a high degree of expertise from patients, caregivers and healthcare providers. Because of the health consequences of poorly treated diabetes and the difficulties experienced by the patients in maintaining healthy blood glucose levels, significant effort has been directed toward automated control of blood glucose concentration.

In recent years, in particular, technological advances in rapid-acting insulin analogs, glucose sensing devices, insulin infusion mechanisms together with the advent of smartphones has led to the development of artificial pancreas (AP) systems, realizing closed-loop control of blood glucose. However, in spite of these advances, around 72% of people requiring exogenous insulin rely upon multiple daily injections (MDIs) of insulin and a finite number of self-monitoring blood glucose measurements (SMBGs) per day. Thus, clinically feasible ways to perform MDI therapy adjustments for both basal insulin delivery and prandial treatments in an automated fashion, learning from sparse glucose measurements and minimal user input are needed to improve T1D patient quality of life and clinical outcomes.

### SUMMARY

The technology described herein relates to control models for insulin injections in people with diabetes, in some cases using artificial pancreases. The methods provided herein allow for a modular and personalized intervention for the treatment of diabetes using an iterative learning controller (ILC). The ILC allows for long-acting insulin doses to be computationally applied to track a basal glucose concentration reference, and/or a run-to-run (R2R) control policy to update the treatment plan, that progressively meets the recommended glycemic targets.

In one aspect, provided herein is a method of updating a basal dose using at least one processor, the method comprising:

determining, using the at least one processor, a tracking error comprising a difference between a measured basal blood glucose concentration and desired basal blood glucose concentration;

determining, using the at least one processor, a basal dose to be administered to a patient based on an Iterative Learning Control (ILC) algorithm wherein using the tracking error; and

storing, in a memory, the basal dose.

In one embodiment of any of the aspects, the method further comprising administering the basal dose to a patient.

In another embodiment of any of the aspects, the ILC algorithm is iterated until a desired threshold is reached.

In another embodiment of any of the aspects, the desired threshold comprises a convergence.

In another embodiment of any of the aspects, the desired threshold comprises a minimization of the tracking error within a threshold window.

In another embodiment of any of the aspects, the method further comprising controlling, using at least one of said at least one processor, the delivery of insulin based on the basal dose.

In another embodiment of any of the aspects, the measured basal blood glucose concentration is determined one, two or three times a day.

In another embodiment of any of the aspects, the tracking error is determined daily, every two days, or every week.

In another embodiment of any of the aspects, the ILC algorithm comprises the formula:

u_{2,j+1}δ(k)=u_{2,j}δ(k)+γQ(q)L(q)ēⱼ(k)
wherein Q represents a zero-phase low pass filter, L is called learning filter, and γ represents a parameter related to a speed of convergence, and ēⱼ(k) is the average error over one iteration of the algorithm.

In another aspect, provided herein is a method of updating a CR value for a Run-to-Run (R2R) controller, using at least one processor, the method comprising:

determining, using the at least one processor, a set of performance parameters based on: a set of preprandial measured and target glucose values; and a set of postprandial measured and target glucose values; and updating, using the at least one processor, a CR profile for a patient preprandial insulin dose to be administered to a patient based on an iterative R2R algorithm using the set of performance parameters; and storing, in a memory, the updated CR profile.

In one embodiment of any of the aspects, the method further comprising receiving a set of data related to a meal from the patient, determining a preprandial dose based on the set of data and the updated CR profile, and administering the preprandial dose to the patient prior to the meal.

In another embodiment of any of the aspects, the patient has type 1 diabetes.

In another embodiment of any of the aspects, the method further comprising controlling, using at least one of said at least one processor, the delivery of insulin based on the preprandial dose.

In another embodiment of any of the aspects, the iterative R2R algorithm comprises an update law according to the formula for a set of j iterations:

$$C {R}_{j + 1}^{m} = \{ \begin{matrix}C {R}_{j}^{m} & if & {y}_{j} ( {k}_{p τ e}^{m} ) > {T}_{b g} ( {k}_{p τ e}^{m} ) \\ & and & {y}_{j} ( {k}_{p o s t}^{m} ) < {T}_{b g} ( {k}_{p o s t}^{m} ) \\ C {R}_{j}^{m} - ( {c}_{1} {G}_{1} + {c}_{2}^{m} {G}_{2} ) & if & {y}_{j} ( {k}_{p o s t}^{m} ) > {T}_{b g} ( {k}_{p o s t}^{m} )\end{matrix}$$

wherein the subscript m, m∈represents meal type {breakfast, lunch, dinner}, wherein kₚᵣₑ^{m} and kₚₒₛₜ^{m} represent time at which finger-sticks blood glucose samples are drawn, wherein T_{bg}(kₚᵣₑ^{m}) represent the preprandial glycemic target for each meal m at the time of finger-stick, wherein T_{bg}(kₚₒₛₜ^{m}) represent the posprandial glycemic target for each meal m at the time of finger-stick, wherein \tilde{c}₁, [symbol] represent controller gains, wherein c₁=\tilde{c}₁·k̆ and [symbol] = [symbol] ·k̆, and wherein k̆ is a patient-specific constant, and wherein G₁ and G₂ represent performance metrics determined by the formulae comprising:

$${G}_{1} = \frac{{y}_{j} ( {k}_{p r e}^{m} ) - {T}_{b g} ( {k}_{p r e}^{m} )}{{T}_{b g} ( {k}_{p r e}^{m} )} and {G}_{21} = \frac{{y}_{j} ( {k}_{p o s t}^{m} ) - {T}_{b g} ( {k}_{p o s t}^{m} )}{{T}_{b g} ( {k}_{p o s t}^{m} )}$$

In another embodiment of any of the aspects, the set of j iterations continues until measured preprandial and post prandial glucose fluctuations are within a threshold.

In another aspect, provided herein is an artificial pancreas for insulin delivery, the artificial pancreas comprising:

at least one non-transitory memory operable to store program code;

an Iterative Learning Control (ILC) including at least one processor operable to read said program code and operate as instructed by said program code, said program code causing the at least one processor to:

determine a set of performance parameters based on a set of measured and target glucose values on a periodic basis; and

update a set of parameters of the ILC based on the set of performance parameters; and

store, in the at least one non-transitory memory, the set of parameters to output an updated ILC; and

deliver the insulin based on the updated ILC.

In one embodiment of any of the aspects, the set of measured and target glucose values comprise basal glucose values.

In another embodiment of any of the aspects, the set of measured and target glucose value further comprise preprandial and postprandial glucose values.

In another embodiment of any of the aspects, said updating the set of parameters of the ILC comprises updating a CR value.

In another embodiment of any of the aspects, said updating the set of parameters of the ILC comprises update a tracking error.

In another embodiment of any of the aspects, the artificial pancreas further comprising a display.

In another embodiment of any of the aspects, the display outputs a suggested dose of insulin to a delivery device and/or a subject.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 demonstrates the compartment model of insulin subsystem provided herein. Left Rapid-acting insulin; Right Long-acting insulin. x₁ and x₂ [pmol/kg] are the amounts of non-monomeric and monomeric, respectively, rapid-acting insulin in the subcu-taneous space; x₃ and x₄ [mU/kg] are the masses of insulin glargine in precipitate and soluble state, respectively; x₅, x₇ and x₆, x₈ [pmol/kg] are rapid- and long-acting insulin, respectively, in plasma and in the liver. Total plasma insulin concentration after injection of rapid-acting u₁ and long-acting u₂ insulin is I=(x₅+x₇)/V_{I}.

FIG. 2 shows controller performance analysis: Top ILC; Bottom R2R. Scenario A (black square), Scenario B (gray plus), Scenario C (cross).

FIG. 3 shows patient #5, Scenario A: blind CGM profile [mg/dl] vs Time [h]. Last day of week 1 (Black), week 10 Red and week 20 Green.

FIG. 4 shows performance analysis for the three considered scenarios based on a blind CGM analysis: CVGA plots. Each marker point represents the coordinates associated to a single patient: Magenta triangle during Week 1; Black square: during Week 20. Each dot and circle represent mean and standard deviation, respectively: Cyan: during the first week in open loop; Blue during the last week of the DSS.

FIG. 5 shows Scenario C. Illustration of the MDI therapy optimization procedure for the in-silico patient #5. Top Daily SMBG samples [mg/dl] grouped by simulation week: Blue maximum, Red average and Black minimum value collected. Center Sizes of daily meal carbohydrate [g] ingested by the in-silico patient grouped by simulation week: Blue breakfast, Yellow lunch and Green dinner. Bottom Basal insulin doses [U]: purple squares indicate the daily dose taken per week; CRs: Red triangle breakfast, Blue diamond lunch and Green dot dinner. Each marker indicates the daily ratio per week.

FIG. 6 shows mean standard deviation of simulated long-acting insulin obtained after an 8-day policy of once-daily subcutaneous administration of Gla-100 with the model in FIG. 1.

FIG. 7 shows a robustness analysis. Nyquist diagrams: L+ΔL (dark gray); L ΔL (black) and circle of unitary radius centered in 1 (light gray).

FIG. 8 demonstrates a compartment model of subcutaneous insulin absorption used in the methods provided herein. The amounts of the injected rapid-acting and long-acting insulin are denoted by u₁ and u₂, respectively. The rate parameters for long-acting absorption are a scaled version of that of rapid-acting absorption.

FIG. 9 shows simulation results using the proposed model starting from steady state with u₁=0, u₂=1 [U]. Top SC insulin [U]; Center Plasma Insulin concentration [pmol/l] and Bottom BG [mg/dl]. Traces show the 10 in-silico patients.

FIG. 10 shows the model approximation with gamma distribution functions for α=3, β=0.16. Traces show the 10 in-silico patients.

FIG. 11 shows simulation scenario 1. Top solid lines denote mean BG [mg/dl] over the 10 in-silico patients; dash-dotted lines denote mean BG±standard deviation σ [mg/dl] Bottom triangles denote mean long-acting insulin doses [U]; dots denote mean doses±σ [U]. ILC (black) open-loop (gray).

FIG. 12 shows simulation scenario 2. Representative Patient 2. Top Daily min-max BG [mg/dl] Bottom Long-acting insulin doses [U]. ILC (black), open-loop (gray).

FIG. 13 shows convergence analysis. Average root mean squared error [mg/dl] vs. iteration number. Fasting (black squares), meals nominal case (stars) and meals with induced insulin resistance (dots).

FIG. 14 shows simulation scenario 2. Three meals a day with induced insulin resistance. Top solid lines denote mean BG [mg/dl] over the 10 in-silico patients; dash-dotted lines denote mean BG±standard deviation σ [mg/dl] Bottom triangles denote mean long-acting insulin doses [U]; dots denote mean doses±σ [U]. ILC (black), open-loop (gray).

FIG. 15 shows gamma distribution and model approximation for the actual dynamics of P(q).

FIG. 16 shows the tracking error and convergence.

FIG. 17 shows a timeline of Scenario 1 and Scenario 2 protocols for meals per day. See also FIGS. 11-14 above.

FIG. 18 shows a table showing the calculation of λ.

FIG. 19 shows a schematic representation of a support system that exploits dynamics and controls concepts.

FIG. 20 depicts an overview of an example system to implement an insulin dose management system according to the present disclosure.

FIG. 21A depicts a flow chart of an example method to implement a basal insulin dose management system according to the present disclosure.

FIG. 21B depicts a flow chart of an example method to implement a preprandial insulin dose management system according to the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

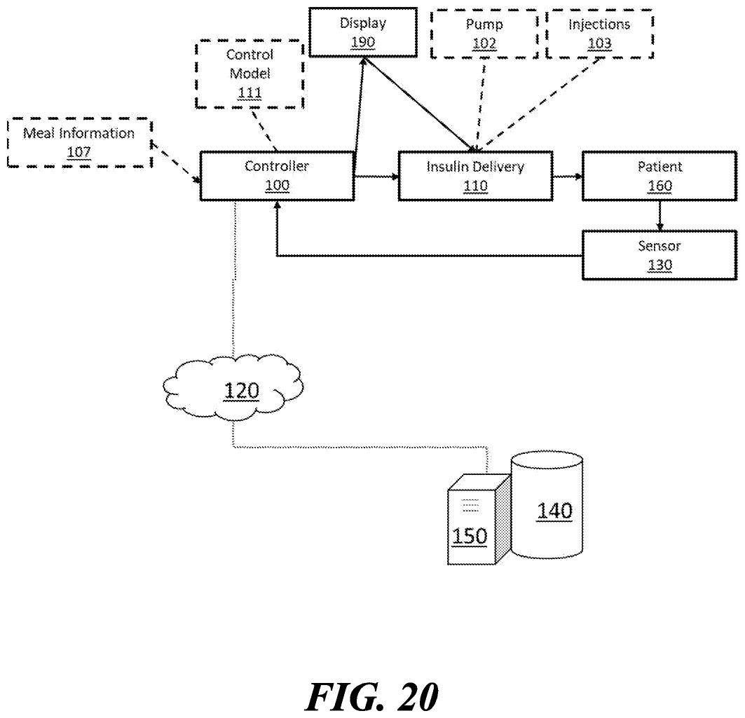

FIG. 20 illustrates an overview of an example system for implementing the disclosed technology. For instance, the system may include a controller 100 that determines insulin bolus amounts to deliver to a patient 160. In some examples, the controller 100 may be connected to a display 190 and may display various insulin dosage amounts so that a patient may manually inject 103 the dosages as directed. In other examples, the controller 100 may provide instructions to a pump 102 to provide insulin boluses to a patient 160 directly through an artificial pancreas, or to automatically fill a syringe or other canister for injection 103 by the patient 160.

The controller 110 may include a control system that has one or more processors, memory and may include or more control models 111, stored on a memory, that process glucose data output from a sensor 130 and/or input by a patient, meal information and data 107, and other data to determine a bolus size of insulin that needs to be delivered to the patient 160. Meal information and data 107 may data relating to various nutritional aspects and quantity of a meal including an amount of carbohydrate content of their meals, for instance the weight of carbohydrate content of a meal (e.g. grams).

The controller 110 may be in communication with a pump 102 and/or display 190 by a wired or wireless connection. Additionally, the glucose sensor 130 may be any suitable sensor for glucose monitoring. In some examples, the glucose sensor 130 may be integrated into a self-administered finger prick test by a patient. In other examples, the glucose sensors 130 may be an under the skin sensor with a wireless connection to the controller 100. In other examples, it may be a non-invasive sensor 130 and have a wired or wireless connection to the controller 100, for instance the FreeStyle Libre manufactured by Abbott Laboratories.

The pump 103 may be any suitable insulin pump that is capable of receiving instructions from the controller 100 and delivering insulin boluses to the patient 160 or to a canister for injection 103 by the patient. For instance, the Medtronic MiniMed 670G is an artificial pancreas using a closed-loop system that includes an insulin pump.

The controller 100 may include various iterative control models 111 for determination of basal and preprandial insulin doses as described herein. This may include adaptation of a basal insulin does of a patient and the insulin-to-carbohydrate ratio (“CR profile” herein), a parameter representing the number of grams of carbohydrate covered by one unit of rapid-acting insulin for delivering preprandial insulin doses that is a function of the body weight of a patient.

The controller 100 may also be connected over a network 120 to a server 150 and a database 140. In some examples, various calculations and model 111 processing will be carried out on local processors on the controller 100 and save on local memory. In other examples, the calculations could be carried out on a server 150 or other computing device in communication with the controller 100.

### Basal Dose

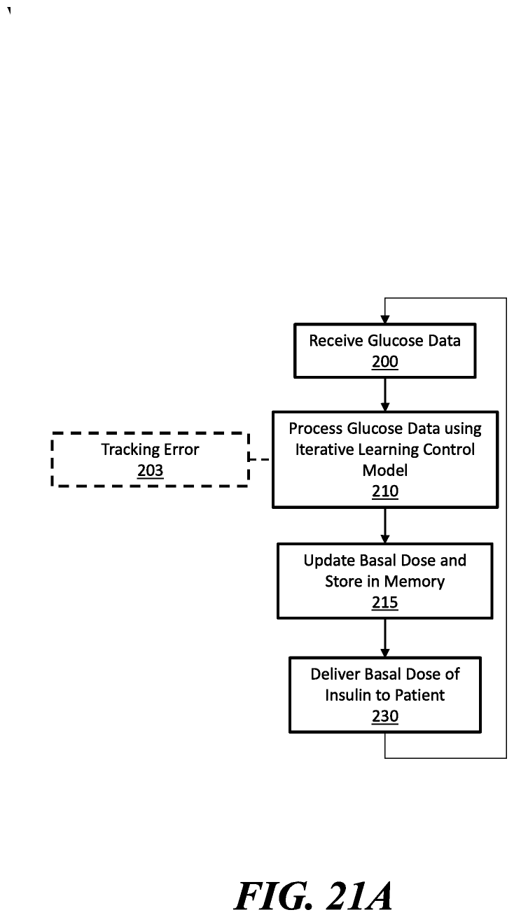

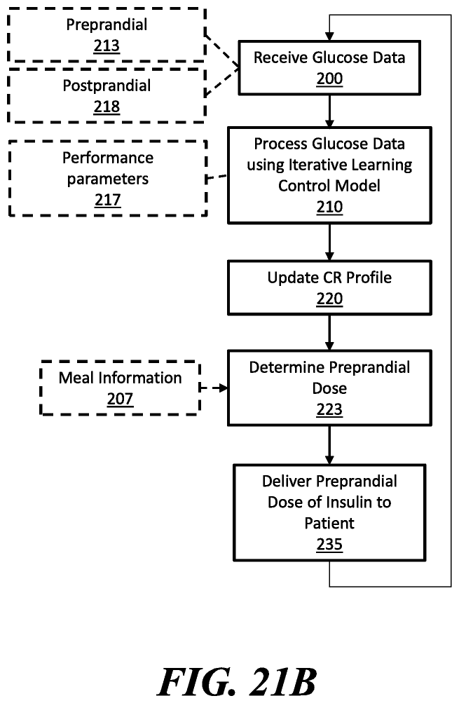

FIG. 21A illustrates an example method for implementing the presently disclosed technology to update a basal dose of insulin to be administered to the patient in order to maintain a patient's basal glucose concentration at a reference level. A basal dose of insulin may include a long acting type of insulin (e.g. Insulin glargine, degludec, or detemir) delivered (e.g. injected) once or twice a day to maintain glucose homeostasis during fasting periods. In some examples, the basal dose rate is updated daily, weekly, monthly or other suitable time periods and based on various time periods of past or prior glucose data compared to reference glucose desired value.

For instance, a controller 100 or other control system may receive glucose data 200 output from a glucose sensor 130 or may be manually entered through a user interface. The received data may be periodically received/entered, relatively continuously received, weekly received, daily received, or other suitable time periods of measurements. In some examples, the glucose level data will be stored in a memory in the controller 100 or a database 140.

The glucose data 200 may them be processed using an iterative control model 210. In some examples, the iterative control model 210 may be based on a tracking error 202, or a comparison of a target and measured glucose value. For instance, the tracking error may be the difference between a measured and desired glucose concentration or other suitable comparisons, and may be made based on varying past numbers of comparisons, for instance one day, two days, two weeks, a month, or other suitable time windows of past tracking error 202. The iterative control model may then update a basal insulin dose stored in a memory 215, of a device, mobile phone application, or other electronic storage device that will be delivered once or twice daily to the patient.

Next, the basal insulin dose may be delivered to the patient 230. In some examples, the basal insulin is manually injected to the patient so the information is displayed to the user or programed to the delivery device. In some examples, the basal insulin is automatically injected. In some examples, this may be through an injection 103 by the patient 160 or through an insulin pump or pen 102. In some examples, this may be performed with closed loop control, or the dosages may be displayed on a display 190 and a patient may then deliver the insulin dosage injection 103 manually.

Additionally, the process may be iterated over time using an iterative learning control algorithm to update the basal insulin dose stored in the memory at regular intervals, for instance daily, weekly, monthly or yearly. In some examples, the update process will cease when the tracking error 203 or other performance parameters hit a threshold indicating the optimization or stability of the blood glucose level of the patient during fasting. In some examples, this may be minimization of tracking error 203 or minimizing the difference between a reference glucose level and a measure glucose basal level.

Once an acceptable threshold of stability is reach, the system may cease to update the parameters until a significant decrease in the performance is detected based on the performance parameters moving out of a threshold. For instance, if the basal measured glucose value moves outside of a threshold window around a reference or desired glucose concentration, the control algorithm may be further iterated.

### CR Profile for Preprandial Dose

FIG. 21B illustrates an example method of providing a preprandial dose to a patient using an run-to run (R2R) that updates the CR profile 220. Patients who count the carbohydrate (CHO) content of their meals calculate prandial insulin boluses with a formula based on ratio of the carbohydrate content to the CR profile (i.e., insulin to carbohydrate ratio). Therefore, before every meal, the patient may calculate the amount or grams of carbohydrate in their meal, calculate an insulin does based on the formula that uses the CR profile, and then deliver the insulin bolus. Accordingly, disclosed are system and methods that use an iterative learning control model 210 to update the CR profile over time, as the algorithm learns with data particular to a patient.

For instance, the system will receive glucose data 200. The glucose data may include preprandial 213 and postprandial 218 glucose data. Preprandial data 213 may be detected prior to a meal, and postprandial 218 glucose data may be detected 1, 1.5, 2, 3, or other suitable times after a meal is ingested. The data may be received over various a periods of time including before and after meals and checked daily, weekly, monthly, or other suitable time frames. The system may iterate the algorithm based one, two, three pre and post prandial meal data sets, or may utilize pre and post prandial glucose data form multiple days. Next, the system may process glucose data using an iterative learning control model 210 to update the CR profile 220 based on various performance parameters 217 that are based on comparing a target glycemic concentration and a pre and postprandially measured glycemic concentration.

Accordingly, based on the updated CR profile, for the next meal, the patient may enter meal information 207 (e.g. carbohydrate mass of an anticipated meal) into an interface. Next, the control model 111 may determine a preprandial insulin dose 223 to be delivered to the patient 160. Next, the preprandial dose of insulin is delivered to the patient before consuming the meal. In some examples, the recommended dose is displayed to the user or programed to a delivery device. Additionally, a set of preprandial glucose data 218 may be received 200 by the system prior to the meal.

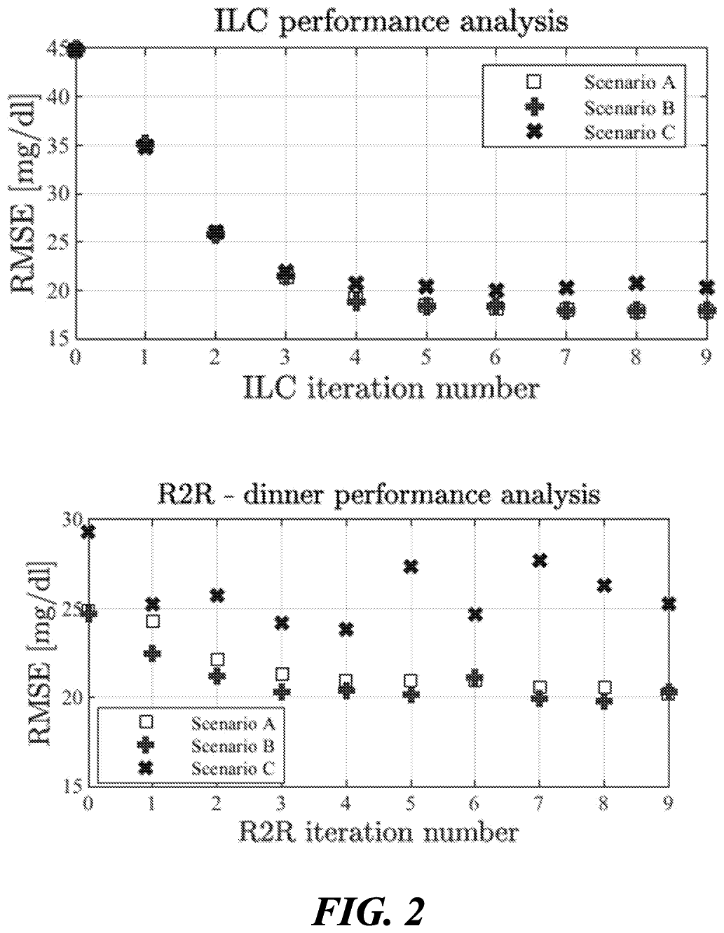

Additionally, postprandial glucose data 218 may then be received 200 by the system after this meal, to iterate the process again to further update the CR profile 220 using the preprandial and post prandial glucose data 218. As discussed with respect to FIG. 2A, the system may then continue to iterate the process until a threshold level of stability is achieved, based on evaluation of performance parameters 217, for instance. In some examples, this may require keeping the blood glucose level of a patient within a certain range after a meal is consumed.

Accordingly, once a threshold level of performance is achieved, the system may monitor the glucose levels 200 to determine whether the performance moves outside a threshold. In those instances, the system may continue to iterate the R2R that updates the CR profile 220.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### EXAMPLES

### Example 1: Using Iterative Learning for Insulin Dosage Optimization in Multiple-Daily-Injections Therapy for People with Type 1 Diabetes

Objective: In this work, iterative algorithms were designed for the delivery of long-acting (basal) and rapid-acting (bolus) insulin, respectively, for people with type 1 diabetes (T1D) on multiple-daily-injections (MDIs) therapy using feedback from self-monitoring of blood glucose (SMBG) measurements.

Methods: Iterative learning control (ILC) updates basal therapy consisting of one long-acting insulin injection per day, while run-to-run (R2R) adapts meal bolus therapy via the update of the mealtime-specific insulin-to-carbohydrate ratio (CR). Updates are due weekly and are based upon sparse SMBG measurements.

Results: Upon termination of the 20 weeks long in-silico trial, in a scenario characterized by meal carbohydrate (CHO) normally distributed with mean μ=[50, 75, 75] grams and standard deviation σ=[5, 7, 7] grams, the strategy provided herein produced statistically significant improvements in time in range (70-180) [mg/dl], from 66.9(33.1) % to 93.6(6.7)%, p=0.02.

Conclusions: Iterative learning shows potential to improve glycemic regulation over time by driving blood glucose closer to the recommended glycemic targets.

Significance: Decision support systems (DSSs) and automated therapy advisors such as the one proposed here are expected to improve glycemic outcomes reducing the burden on patients on MDI therapy.

### 1. Introduction

People with type 1 diabetes (T1D) require exogenous insulin administration for adequate blood glucose regulation [1]. The aim of such insulin therapy is to mimic as closely as possible the physiological insulin secretion pattern in the individual without diabetes consisting in a slow basal secretion throughout the day and an augmented rate at meal times. Insulin treatment is burdensome to the patient, it entails great effort and requires a high degree of expertise from patients, caregivers and healthcare providers. Because of the health consequences of poorly treated diabetes and the difficulties experienced by the patients in maintaining healthy blood glucose levels, significant effort has been directed toward automated control of blood glucose concentration. In recent years, in particular, technological advances in rapid-acting insulin analogs, glucose sensing devices, insulin infusion mechanisms together with the advent of smartphones has led to the development of artificial pancreas (AP) and/or decision support apparatus systems, realizing closed-loop control of blood glucose [2], [3], [4], [5]. In this context, the most common platforms adopted for sensing and actuation, respectively, are continuous glucose monitors (CGM) and continuous subcutaneous insulin infusion (CSII) pumps [2] allowing the controlled variable, i.e., blood glucose level, to be measured with a sampling period of 5 to 15 [min] and the control input, i.e., rapid-acting insulin, to be delivered every 5 or 10 minutes. However, in spite of these advances, around 72% of people requiring exogenous insulin rely upon multiple daily injections (MDIs) of insulin and a finite number of self-monitoring blood glucose measurements (SMBGs) per day. In fact, the reported differences in hemoglobin A1C and severe hypoglycemia rates between intensive insulin therapy by means of MDIs versus CSII [6] favors the first when it comes to factors related to cost and device usability.

Contrary to typical AP systems, the MDI treatment comprises the delivery of two types of insulin formulations: a long-acting analog (e.g., Insulin glargine, degludec or detemir) to maintain glucose homeostasis during fasting by providing a basal insulin concentration and a superimposed rapid-acting analog (e.g., Insulin lispro, aspart or glulisine) at meal times to compensate for the glycemic excursions due to the macronutrient content of the ingested food or as a correction for hyperglycemia [1]. Typically, basal insulin is injected once or twice a day e.g., every morning before breakfast or every night before bedtime. Whereas as far as prandial insulin is concerned, patients counting the amount of carbohydrate in their meals adapt their meal bolus regimen according to the insulin-to-carbohydrate ratio (CR), a parameter representing the number of grams of carbohydrate covered by one unit of rapid-acting insulin. In this treatment modality, patients need to choose, check and change periodically the basal insulin dose and the CRs, the initial long-acting insulin doses and CRs generally being a function of the body weight and revised by incremental adjustments based on guidelines until a desirable level of control is attained [1]. Nevertheless, evidence shows that the majority of people with T1D on either MDI or CSII therapy do not reach the desired glycemic targets. There are compelling reasons, therefore, to study injection policies for long-acting insulin alone and with rapid-acting insulin with the goals of improving patients' quality of life and health outcomes.

Several algorithms for automated adjustments of MDI regimes have been proposed in the literature, ranging from PID and fuzzy logic tuning rules [7], [8], iterative learning strategies [9], [10], [11], [12] and optimization-based approaches [13], [14], [15]. Most of these works focused solely on the prandial boluses calculation, assuming the optimal basal therapy, comprised of one or more long-acting insulin boluses per day, was given.

Against this background, the present contribution outlines clinically feasible ways to perform MDI therapy adjustments for both basal insulin delivery and prandial treatments in an automated fashion, relying upon iterative learning from sparse blood glucose measurements and minimal user input. The choice of learning controllers is motivated by the fact that insulin therapy for people with T1D is repetitive in nature. Moreover, of specific interest, are effective strategies for outpatient use to be deployed as a decision support system (DSS) by the patients in their daily life.

In this approach, the disjoint adjustment of the basal and prandial insulin regimes were explored, respectively, focusing independently on the attainment of a steady unperturbed blood glucose concentration around a reference level of 110 [mg/dl], i.e., the basal blood glucose concentration, with the injection of long-acting analogs once daily, and a prandial glucose concentration in the target range (100-160) [mg/dl], using rapid-acting analogs at mealtime. In particular, the failure to maintain a proper basal glucose concentration will impair every tentative strategy for keeping blood glucose within the euglycemic zone in response to the various disturbances acting on glucose metabolism and hence in the methods provided herein for basal therapy is given uttermost importance.

The methods provided herein are different from existing works in the literature in several aspects. First, the disjoint therapy optimization introduces a new degree of freedom, namely the choice of what therapy parameter needs to be revised at each time, allowing for a modular and more personalized intervention. Second, the control of the basal blood glucose concentration around a steady level was viewed as a reference tracking problem.

That said, an iterative learning controller (ILC) [16], [17], [18], [19] was applied for long-acting insulin doses computation to track a basal glucose concentration reference, and a run-to-run (R2R) [20] control policy to update the treatment plan's CRs, to progressively meet the recommended glycemic targets.

In this contribution, the length of each iteration of the proposed algorithms was set to one week i.e., a new therapy update consisting of either a new long-acting insulin dosage or 3 new mealtime-specific CRs was provided every week and it was calculated exploiting data from the previous week. In current clinical practice, patients may visit their physicians for a review of their treatments more infrequently than what assumed herein. The methods introduced in this paper, however, apply mutatis mutandis irrespective of the length of each run. The proposed strategy was tested on a population of 10 in-silico MDI patients and show that the revised injection policy produces desired glucose regulation not only in the nominal case, but also in presence of variations in meal sizes and sampling schedule.

The layout of the remainder of the paper is as follows: in Section 2 a description of the model used for the in-silico population is provided, Sec. 3 outlines the iterative learning controller design, Sec. 4 introduces the run-to-run controller, Sec 5. gives the simulation scenarios for the verification of the recommended treatment policies along with details pertaining the practical implementation of the proposed strategy for a proof-of-concept outpatient study while Sec. 6. presents results obtained using the strategy provided herein on the simulated patients and finally in Section 7., discussion of the findings conclusions are provided.

### 2. Metabolic Model of Virtual Patient

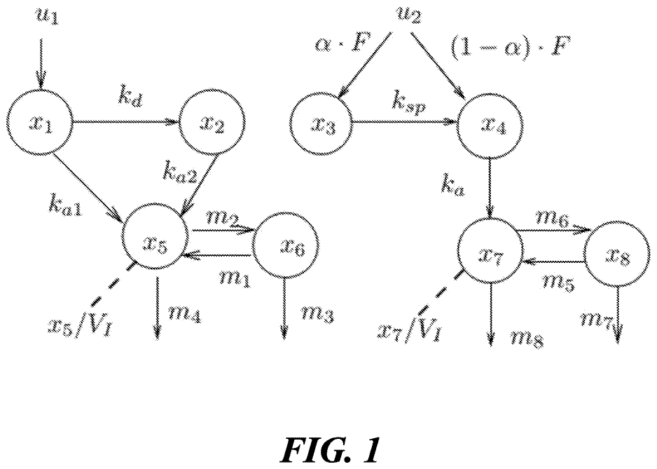

In-silico simulations facilitate the synthesis of control algorithms and their verification under several experimental conditions prior to their deployment in human clinical trials. Thus, for the purpose of testing feasibility and effectiveness of the proposed controllers, a mathematical model of the glucose-insulin metabolic system was able to simulate MDI treatment in people with T1D. The model is based on the research first presented in [21], [22], with the incorporation of a module describing the pharmacokinetics (PK) of long-acting basal insulin introduced in [23], [24]. Briefly, with reference to FIG. 1, inputs to the model are rapid-acting u1 and long-acting insulin u₂. The subcutaneous insulin kinetics for rapid-acting insulin is described as a chain of two compartments, where the first compartment represents insulin in the non-monomeric state while the second compartment represents insulin in the monomeric state [21], [22].

As for long acting insulin, the first and second compartment represent insulin glargine (Gla-100) in precipitate and soluble state, respectively [24], [25]. Provided herein is a model of insulin in plasma and its degradation in the liver and periphery, for each type of insulin, as a chain of two compartments [22] and propose that the total plasma insulin concentration l(t) [pmol/l] be as:

I(t)=(x₅(t)+x₇(t))/V_{I} (1)

where x₅ and x₇ [pmol/kg] are rapid- and long-acting insulin, respectively, in plasma, V₁ [l/kg] is the volume of insulin distribution which it was assumed to be the same for both rapid-acting and long-acting analogs once they reach plasma. For simplicity, initial conditions for all the insulin states were set to zero i.e., xᵢ (0)=0, i∈{1, . . . 8}. In addition, the metabolic model includes a gastro-intestinal subsystem which describes digestion and absorption of the carbohydrate content of a meal and subsequent rate of appearance of glucose in blood; a glucose subsystem describing insulin-independent utilization in plasma and fast-equilibrating tissue, and insulin-dependent utilization in the periphery and endogenous glucose production.

Parameter values pertaining gastro-intestinal subsystem, glucose subsystem, endogenous glucose production, subcutaneous and plasma rapid-insulin kinetics from the 10 in-silico adults reported in [22] were used to create the simulated patients used in this contribution. Insulin-to-carbohydrate ratios were obtained by randomly adding or subtracting a number normally distributed, with mean 1 and standard deviation 0.5 to the values given in [22]. Such parameter set was augmented with parameters relative to the Gla-100 model randomly drawn from their empirical distribution and randomly assigned to each in-silico subject. More details on model equations and parameters are given in EXAMPLE 2: Appendix.

### 3. Iterative-Learning-Control (ILC) for Basal Insulin Therapy

People with T1D inject long-acting insulin every day in the same conditions with the goal of providing a background insulin concentration and hence maintaining their basal glucose concentration at a reference level. The tracking error calculated day-by-day or week-by-week can therefore be considered repeatable and can be used to adjust the long-acting insulin dosing for the next day or week in an automated way, in order to progressively improve the reference following, assuming some knowledge about the system describing glucose excursions in response to basal therapy is available. This approach lends itself naturally to the application of ILC.

ILC has found successful application in a variety of reference tracking scenarios (see e.g., [19] and references therein). A favorable advantage of ILC is that high tracking performances are achieved without the requirement of an accurate mathematical model of the underlying system dynamics. This feature is particularly appealing in the case at hand, given the uncertainties in the basal insulin PK/pharmacodynamics (PD) and the large inter- and intra-patient variability.

That said, let the blood glucose excursion due to a bolus of long-acting insulin during fasting conditions be described by a discrete-time, linear-time-invariant (LTI), single input-single output (SISO) system:

yⱼ^{b}=P(q)u_{2,j}δ(k) (2)

where y^{b} denotes basal blood glucose concentration, u₂ long-acting insulin, j is the iteration index, k the discrete-time index, δ is the delta function and q the delay operator. Note that for simplicity it is assumed that no external disturbances, such as emotional stress, intercurrent illness, hormonal variations and physical activity, are acting on the system. The tracking error i.e., the difference between the measured and the desired basal blood glucose concentration r(k) is defined as:

eⱼ(k)=r(k)−yⱼ^{b}(k) (3)

The ILC algorithm presented herein finds the dose u_{2,j}δ(k) to be administered such that the measured basal blood glucose concentration is driven as close as possible to the desired reference trajectory r(k) by using an iterative method:

u_{2,j}+1δ(k)=u_{2,j}δ(k)+γQ(q)L(q)eⱼ(k) (4)

where Q is a zero-phase low pass filter, L is called learning filter, γ is a parameter related to speed of convergence and eⱼ(k) is the average error over one iteration of the algorithm. Note that the filters Q and L can be either causal or non-causal as they operate on signals from the previous iteration of the algorithm. Various methods have been proposed in the literature for the design of L (see e.g., [19], [20]). Here a model-based approach as in [12] was used, in which the inverse of an approximate model \hat{P}(s) of the actual dynamics P(s), with s the Laplace operator, between long-acting insulin injection and blood glucose is used as learning filter:

$$\hat{P} ( s ) = k \frac{1}{{( 375 s + 1 )}^{3}} \qquad ( 5 )$$

Details on the approximation \hat{P}(s) with gamma distribution functions of the actual dynamics P(s) used in this Example are reported in [12]. After discretization of L(s) and Q(s) with the zero-order-hold (ZOH) method, the following expression for the tracking error is derived:

eⱼ₊₁(k)=(1−P(q)QP⁻¹(q))·eⱼ(k) (6)

Convergence is achieved if

|1−P(e^{iωt}^{ₛ})Q(e^{iωt}^{ₛ})\hat{P}⁻¹(e^{iωt}^{ₛ})|<1 (7)

where ω∈[−π, π]. The choice of Q affexts the robustness of the algorithm and the speed of convergence and is dictated by the relative rerror between the approximation \hat{P} and the true P. In order to eliminate high frequency plan-model mismatches and to fulfill the condition in (7) for the in-silico population, the equation below was chosen.

$$Q ( s ) = \frac{1}{{( 60 s + 1 )}^{2}} \qquad ( 8 )$$

More details on the robustness of ILC formulation are provided in EXAMPLE 2. In this setting, at each iteration j of the algorithm, the SMBG preprandial measurements were taken 3 times a day, before breakfast, lunch and dinner as the measured basal blood glucose concentration y_{b}(k) used to compute the tracking error.

### 4. Run-to-Run (R2r) Control for Meal Bolus Insulin Therapy

Patients who count the carbohydrate (CHO) content of their meals, calculate prandial insulin boluses with the formula [25], [26]:

$${u}_{1} [ U ] = \frac{C H O [ g ]}{C R} \qquad ( 9 )$$

Various methods have been proposed in the clinical literature for the quantification of the CR profile according to time of day (e.g., [25]) on the basis of the time-varying nature of insulin sensitivity. The design objective is to compute three CRs, each corresponding to breakfast, lunch, and dinner, starting from a given initial value.

Similar to what is discuss in Section 3. above, an iterative paradigm with a learning-type controller was introduced to update the CRs. The R2R control strategy [20] presented in this section is an iteration-based approach that has been applied in the diabetes control field in several works [9], [10], [27], [28], [29], [30]. Within this framework, information about the glycemic outcomes measured by clinically relevant performance metrics was used based on sparse glucose samples from the past iteration to change the CRs for the next iteration, in order to progressively meet the recommended glycemic targets. This is distinctly different from the ILC-based basal therapy controller in Section 3. whose control objective was to track an unperturbed basal glucose concentration reference. Further, as opposed to previously proposed R2R-based controller which provided the amount of insulin to be injected as control input, the aim was to suggest a CR value. Changing this patient parameter provided more flexibility in the treatment, since it allows the adaption of the meal bolus based on the amount of ingested carbohydrate.

Given the glycemic goals for preprandial and peak post-prandial capillary glucose outlined in [1], at each iteration j, the algorithm proposed here prescribes an update law for the CRs to be applied to the next iteration j+1 as follows:

$$C {R}_{j + 1}^{m} = \{ \begin{matrix}C {R}_{j}^{m} & if & {y}_{j} ( {k}_{pre}^{m} ) > {T}_{b g} ( {k}_{pre}^{m} ) \\ & and & {y}_{j} ( {k}_{p o s t}^{m} ) < {T}_{b g} ( {k}_{p o s t}^{m} ) \\ C {R}_{j}^{m} - ( {c}_{1} {G}_{1} + {c}_{2}^{m} {G}_{2} ) & if & {y}_{j} ( {k}_{p o s t}^{m} ) > {T}_{b g} ( {k}_{p o s t}^{m} )\end{matrix} \qquad ( 10 )$$

wherein the subscript m, m∈represents meal type {breakfast, lunch, dinner},
wherein kₚᵣₑ^{m} and kₚₒₛₜ^{m} represent time at which finger-sticks blood glucose samples are drawn,
wherein T_{bg}(kₚᵣₑ^{m}) represent the preprandial glycemic target for each meal m at the time of finger-stick,
wherein T_{bg}(kₚₒₛₜ^{m}) represent the posprandial glycemic target for each meal m at the time of finger-stick,
wherein \tilde{c}₁, [symbol] represent controller gains, wherein c₁=\tilde{c}₁·k̆ and [symbol] = [symbol] ·k̆, and wherein
k̆ is a patient-specific constant,
and wherein G₁ and G₂ represent performance metrics determined by the formulae comprising:

$$\begin{matrix}{G}_{1} = \frac{{y}_{j} ( {k}_{pre}^{m} ) - {T}_{bg} ( {k}_{pre}^{m} )}{{T}_{bg} ( {k}_{pre}^{m} )} , and & ( 11 ) \\ {G}_{21} = \frac{{y}_{j} ( {k}_{p o s t}^{m} ) - {T}_{bg} ( {k}_{p o s t}^{m} )}{{T}_{bg} ( {k}_{p o s t}^{m} )} & ( 12 )\end{matrix}$$

As a result of overbolusing a meal m−1, the capillary glucose sample yⱼ(kₚᵣₑ^{m}) taken prior to the next meal m, may be below the recommended target T_{bg}(kₚᵣₑ^{m}), i.e., yⱼ(kₚᵣₑ^{m})<T_{bg}(kₚᵣₑ^{m}). In that circumstance, the CR for the previously overbolused meal is increased:

CRⱼ₌₁^{m-1}=CRⱼ^{m-1}+c₁G₃ (13)

with \tilde{c}₃=\tilde{c}₃·k̆ and G₃ performance metric:

$${G}_{3} = \frac{{T}_{bg} ( {κ}_{pre}^{m} ) - {y}_{j} ( {k}_{pre}^{m} )}{{T}_{bg} ( {k}_{pre}^{m} )} \qquad ( 14 )$$

The algorithm terminates when convergence is achieved or when a satisfactory degree of control, evaluated with the criteria below is attained:

$$\begin{matrix}min ( {y}_{j} ( {k}_{pre}^{m} ) ) & > {\hat{s}}_{b g} \\ max ( {y}_{j} ( {k}_{p o s t}^{m} ) ) & < {{s}^{⋁}}_{b g} \\ \frac{min ( {y}_{j} ( {k}_{pre}^{m} ) ) + max ( {y}_{j} ( {k}_{p o s t}^{m} ) )}{2} & < {s}_{b g}\end{matrix} \qquad ( 15 )$$

where ŝ_{bg}, š_{bg}, s_{bg} are user-defined thresholds.

TABLE 1
Summary of Updating Rules for CR

| Condition | Rule |
|---|---|
| yⱼ(kₚᵣₑ^{m}) < T_{bg} (kₚᵣₑ^{m}) | CRⱼ₊₁^{m-1} = CRⱼ^{m-1} + c₁G₃ |
| yⱼ(kₚᵣₑ^{m}) > T_{bg} (kₚᵣₑ^{m}) & | CRⱼ₊₁^{m} = CRⱼ^{m} |
| yⱼ(kₚₒₛₜ^{m}) < T_{bg}(kₚₒₛₜ^{m}) | CRⱼ₊₁^{m-1} = CRⱼ^{m} − (c_{1,j}G₁ + c_{2,j}^{m}G₂) |
| yⱼ(kₚₒₛₜ^{m}) > T_{bg}(kₚₒₛₜ^{m}) | |

### 5. In-Silico Performance Analysis

The proposed ILC and R2R strategies were evaluated in simulation using the metabolic model outlined in Section 2. Albeit small, the use of this population provided a means to test the algorithms robustness with respect to inter-subject variability. The in-silico protocol was motivated by and designed for the clinical translation of the proposed MDI treatment in future clinical trials. The case-study simulations started at 7 am on Day 1 and lasted 21 weeks, finishing at 6:59 am on Day 147. the first week was an open-loop, run-in week needed to let the transients in the metabolic model extinguish and the injected insulin stack up, given that simulations started with zero initial conditions. Thus, zero initial conditions is referred to herein as simulation Week 0 and it is not considered in the analysis of performances. At 7 am on Day 8, the actual simulation study Week 1 commenced. Weeks 1 to 10 after the run-in week were used to test the ILC, keeping the CRs constant throughout, whereas the subsequent 10 weeks, i.e., 11 to 20, evaluated the R2R strategy, maintaining as basal therapy the ILC-determined optimal therapy upon convergence. The chronological order of the treatment optimization as well as the disjoin adjustment of basal/bolus therapy followed standard clinical practice. In all test cases, Week 1 was in open-loop and the simulated patients complied with conventional therapy, blood glucoses initial condition was y(0)=180 [mg/dl], initial treatment parameters were basal insulin dose u₂(0)=0.4 [U] per kg of body weight and CRs reported in Table 2.

Three simulation scenarios were considered:

Scenario A (nominal case). Meals were taken a 7 am, 1 pm, 7 pm each day and the amount of carbohydrate per meal was 50 [g], 75 [g], and 75 [g], respectively. SMBG samples were drawn preprandial at 7 am, 1 pm, and 7 pm, respectively and 2-h postprandial at 9 am, 3 pm, and 9 pm respectively, each day.

Scenario B (robustness against disturbances). Meal times and SMBG inputs were identical to Scenario A and the amount of carbohydrate per meal was normally distributed with meals (50, 75, 75,) [g] and standard deviations (5, 7, 7) [g], respectively. Self-monitoring blood glucose samples were drawn as in Scenario A.

Scenario C (effect of systematic error in SMBG timing). Meal times and amount of carbohydrate were as per Scenario A. The timing of self-monitoring blood glucose samples were perturbed by adding or subtracting a randomly sampled normally distributed bias with mean 20 min and standard deviation 15 minutes from the timing in Scenario A.

TABLE 2
Treatment Parameters for the 10 In-Silico Patients:
Initial (Subscript 0) Vs. Final (Superscript *)

| Sc | # | u_{2,}0 | u^{*}_{2,} [u] | CR₀ [WU] | CR^{*,b} [g/U] | CR^{*,l}[g/U] | CR^{*,d}[g/U] |
|---|---|---|---|---|---|---|---|
| | 1 | 31 | 31 | 10.6 | 11.1 | 7.1 | 9.4 |
| | 2 | 41 | 24 | 12.2 | 9.1 | 13.7 | 12.5 |
| | 3 | 30 | 30 | 6.2 | 3.0 | 4.7 | 3.0 |
| | 4 | 23 | 39 | 14.5 | 14.3 | 15.0 | 15.0 |
| | 5 | 24 | 29 | 11.7 | 7.7 | 9.4 | 8.3 |
| | 6 | 27 | 27 | 11.4 | 11.1 | 11.5 | 11.5 |
| | 7 | 27 | 23 | 14.9 | 14.3 | 15.0 | 15 |
| | 8 | 27 | 20 | 13.4 | 12.5 | 13.6 | 13.6 |
| | 9 | 26 | 39 | 7.8 | 7.7 | 7.9 | 7.9 |
| | 10 | 27 | 30 | 11.4 | 11.1 | 11.5 | 11.5 |
| | μ(σ) | 28.3(5.0) | 29.2(6.2) | 11.4(2.7) | 10.1(3.4) | 10.9(3.5) | 10.8(3.8) |
| Scenario | 1 | 31 | 31 | 10.6 | 11.1 | 6.0 | 9.4 |
| B | 2 | 41 | 23 | 12.2 | 12.5 | 12.5 | 12.5 |
| | 3 | 30 | 30 | 6.2 | 3.0 | 4.4 | 4.0 |
| | 4 | 23 | 38 | 14.5 | 14.2 | 15.0 | 15.0 |
| | 5 | 24 | 29 | 11.7 | 7.7 | 10.0 | 7.9 |
| | 6 | 27 | 28 | 11.4 | 11.1 | 11.5 | 11.5 |
| | 7 | 27 | 23 | 14.9 | 14.3 | 15.0 | 15.0 |
| | 8 | 27 | 20 | 13.4 | 12.5 | 13.6 | 13.6 |
| | 9 | 26 | 39 | 7.8 | 7.7 | 7.9 | 7.9 |
| | 10 | 27 | 30 | 11.4 | 11.1 | 11.5 | 11.5 |
| | μ(σ) | 28.3(5.0) | 29.1(6.1) | 11.4(2.7) | 10.5(3.4) | 10.2(3.5) | 10.8(3.5) |
| Scenario | 1 | 31 | 38 | 10.6 | 11.1 | 10.7 | 10.7 |
| C | 2 | 41 | 24 | 12.2 | 11.1 | 13.6 | 12.5 |
| | 3 | 30 | 43 | 6.2 | 3.0 | 3.9 | 3.0 |
| | 4 | 23 | 41 | 14.5 | 14.3 | 15.0 | 15.0 |
| | 5 | 24 | 37 | 11.7 | 10.0 | 10.7 | 10.0 |
| | 6 | 27 | 31 | 11.4 | 11.1 | 11.5 | 11.5 |
| | 7 | 27 | 27 | 14.9 | 14.3 | 15.0 | 15.0 |
| | 8 | 27 | 21 | 13.4 | 14.3 | 13.6 | 13.6 |
| | 9 | 26 | 46 | 7.8 | 7.7 | 8.9 | 8.9 |
| | 10 | 27 | 35 | 11.4 | 11.1 | 11.5 | 11.5 |
| | μ(σ) | 28.3(5.0) | 34.3(8.3) | 11.4(2.7) | 11.1(3.4) | 11.4(3.3) | 11.1(3.5) |

Values of the controllers gains were γ=1, \tilde{c}₃=1, \tilde{c}₁=1.5, \tilde{c}₃^{m}=1.7 when m∈{breakfast, lunch} and \tilde{c}₃^{m}=1.6 when m=dinner. These were found to be best for the considered population. Individual-specific parameter values were k=BW/3 and k̆=BW·TDD where BW denotes body weight and TDD total daily dose of insulin from the previous week. The reference for basal glucose concentration was r(k)=110 mg/dl, preprandial and postprandial targets, respsectively were T_{bg}(kₚᵣₑ)=100 mg/dl and T_{bg}(kₚₒₛₜ)=160 mg/dl for each meal, the user defined thresholds were ŝ_{bg}=80 mg/dl, š_{bg}=180 mg/dl, s_{bg}=150 mg/dl. To ensure safety, maximum change in dose |Δu₂| between iterations was |Δu_{j+1,j}|=|uⱼ₊₁−uⱼ|≤0.20·uⱼ, where the subscript 2 was dropped for notational convenience, while the allowed change in CR between iterations was |ΔCR_{j+1,j}^{m}|=|ΔCRⱼ₊₁^{m}−CRⱼ^{m}|, 0.5≤|ΔCR_{j+1,j}^{m}|≤4, the minimum allowed CR being 3. Last, in order to accommodate current actuator limitations in the delivery of long-acting and rapid acting insulin, respectively, the resolution of the dose increment was 1 [U] for basal therapy and 0.5 [U] for prandial doses.

### 6. Results

Treatment parameters for the 10 in-silico patients, grouped by scenarios, are reported in Table 2. The highlighted columns list the starting parameters while the remaining columns list the final parameters obtained with the proposed controllers. As far as long-acting insulin in concerned, the optimal doses recommended by the ILC are almost identical in Scenario A and B, meaning that the controller is robust against meal sizes which are randomly sampled from a normal distribution with σ=10% of the original meal sizes. On the contrary, in Scenario C, across all the in-silico patients in the population, the long-acting insulin doses recommended by the ILC are on average 17.8% higher than in the other scenarios. This is due to the fact that in some cases, SMBG samples were taken at times far away from the prescribed fasting conditions. This impacts the performances in that the ILC controller commands more basal dose than actually needed, compensating for an elevated basal glucose concentration due to food consumption. Similarly, the R2R strategy is not significantly affected by changes in meal sizes. In fact, only 20% of the CRs in Scenario B are different from those in Scenario A. As for Scenario C, 30% of the CRs show changes in magnitude compared to the nominal case.

FIG. 2 shows the population average root mean squared error [mg/dl] between the SMBGs and the reference basal blood glucose concentration during weeks 1 to 10 (top) and between the mean SMBGs for dinner and the mean of the corresponding reference, i.e., 130 [mg/dl], during weeks 11 to 20 (bottom). Dinner was chosen as a representative meal. Convergence of the ILC is achieved between iteration #6 and 7, across scenarios, although little to no variation can be seen after iteration #4. Note that the numerical values of the RMSE for the three scenarios are very close. Similar conclusions can be drawn for the R2R algorithm as far as the first two scenarios are concerned. Minor oscillations in the tail are due to actuator limitations in the delivery of rapid-acting insulin, which practically prohibits the delivery of a dose with granularity less than 0.5 [U]. Notice that iteration #0 of the ILC algorithm corresponds to Week 1 of the in-silico protocol, while iteration #0 of the R2R corresponds to Week 10.

Numerical results are tabulated in Table 3.

TABLE 3
Glycemic Outcomes in Week 1, Week 10, Week 20 for the in-silico population:
Mean(Standard deviation), min, max, mean of drawn SMBG Sample; BG% time in Zones of
Blind CGM; Daily long-acting insulin u₂ [U] and average total daily rapid-acting insulin u₁ [U].
p-value ₁₋₁₀ between Week 1 and Week 10, p-value ₁₋₂₀ between Week 1 and Week 20 and p-vlaue 10-
20 between Week 10 and Week 20.

| | Metrics | Week 1 | Week 10 | Week 20 | p-value₁₋₁₀ | p-value₁₋₂₀ | p-value₁₀₋₂₀ |
|---|---|---|---|---|---|---|---|
| Scenario A | min [mg/dl] | 95.2(50.8) | 92.9(12.1) | 91.1(9.4) | 0.87 | 0.71 | 0.29 |
| | max [mg/dl] | 183.6(71.3) | 174.1(28.5) | 170.2(23.6) | 0.70 | 0.65 | 0.36 |
| | mean [mg/dl] | 139.7(54.3) | 136.5(16.1) | 132.5(12.0) | 0.83 | 0.65 | 0.08 |
| | % time ∈ (70,180) | 66.8(33.8) | 90.5(10.5) | 94.6(6.8) | 0.05 | 0.03 | 0.04 |
| | [mg/dl] | | | | | | |
| | % time <70 [mg/dl] | 14.1(30.7) | 1.6(5.2) | 0.0(0.0) | 0.17 | 0.17 | 0.34 |
| | % time <54 [mg/d1] | 9.7(23.6) | 0.0(0.0) | 0.0(0.0) | 0.22 | 0.22 | NaN |
| | % time >180 [mg/d1] | 19.1(28.3) | 7.9(10.6) | 5.4(6.8) | 0.25 | 0.16 | 0.07 |
| | % time >250 [mg/d1] | 2.8(8.8) | 0.0(0.0) | 0.0(0.0) | 0.34 | 0.34 | NaN |
| | u₂ [U] | 28.3(5.0) | 29.2(6.2) | 29.2(6.2) | — | — | — |
| | u₁ [U] | 18.6(5.8) | 18.6(5.8) | 22.3(13.0) | — | — | — |
| | u₂ : u₁ | 1.5(0.9) | 1.5(1.0) | 1.3(0.5) | — | — | — |
| Sc | min [mg/dl] | 99.1(49.5) | 89.3(14.8) | 86.7(10.9) | 0.72 | 0.72 | 0.86 |
| en | max [mg/dl] | 188.9(72.3) | 182.5(31.0) | 175.0(23.2) | 0.79 | 0.81 | 0.80 |
| | mean [mg/dl] | 139.1(54.1) | 136.4(15.9) | 133.2(10.6) | 0.84 | 0.72 | 0.21 |
| | % time ∈ (70,180) | 66.9(33.1) | 90.3(10.5) | 93.6(6.7) | 0.05 | 0.02 | 0.04 |
| | [mg/dl] | | | | | | |
| | % time <70 [mg/dl] | 14.5(31.3) | 1.5(4.5) | 1.0(3.3) | 0.17 | 0.17 | 0.28 |
| | % time <54 [mg/d1] | 9.9(23.9) | 0.3(1.0) | 0.0(0.0) | 0.21 | 0.22 | 0.34 |
| | % time >180 [mg/d1] | 18.5(26.7) | 8.2(10.6) | 5.4(6.3) | 0.26 | 0.15 | 0.09 |
| | % time >250 [mg/d1] | 2.9(9.1) | 0.0(0.0) | 0.0(0.0) | 0.34 | 0.34 | NaN |
| | u₂ [U] | 28.3(5.0) | 29.1(6.1) | 29.1(6.1) | — | — | — |
| | u₁ [U] | 18.5(5.7) | 18.9(5.9) | 22.5(12.7) | — | — | — |
| | u₂ : u₁ | 1.5(0.9) | 1.5(1.0) | 1.3(0.5) | — | — | — |
| Scenario C | min [mg/dl] | 100.5(50.2) | 87.5(5.3) | 87.7(7.1) | 0.27 | 0.18 | 0.16 |
| | max [mg/dl] | 194.4(77.4) | 162.7(24.6) | 159.1(18.7) | 0.56 | 0.46 | 0.11 |
| | mean [mg/dl] | 145.9(55.9) | 130.3(12.4) | 125.7(11.2) | 0.35 | 0.27 | 0.15 |
| | % time ∈ (70,180) | 67.2(32.8) | 92.3(9.7) | 93.8(7.0) | 0.03 | 0.02 | 0.1 |
| | [mg/dl] | | | | | | |
| | % time <70 [mg/dl] | 14.7(31.6) | 3.7(7.5) | 4.0(7.0) | 0.23 | 0.27 | 0.87 |
| | % time <54 [mg/d1] | 10.1(24.2) | 0.7(2.3) | 0.4(1.2) | 0.21 | 0.21 | 0.34 |
| | % time >180 [mg/d1] | 18.2(25.9) | 3.9(8.4) | 2.1(4.4) | 0.10 | 0.07 | 0.16 |
| | % time >250 [mg/d1] | 2.9(8.9) | 0.0(0.0) | 0.0(0.0) | 0.33 | 0.33 | NaN |
| | u₂ [U] | 28.3(5.0) | 34.3(7.9) | 34.3(7.9) | — | — | — |
| | u₁ [U] | 16.7(7.8) | 17.2(7.9) | 19.7(15.4) | — | — | — |
| | u₂ : u₁ | 1.7(0.6) | 2.0(1.0) | 1.7(0.5) | — | — | — |

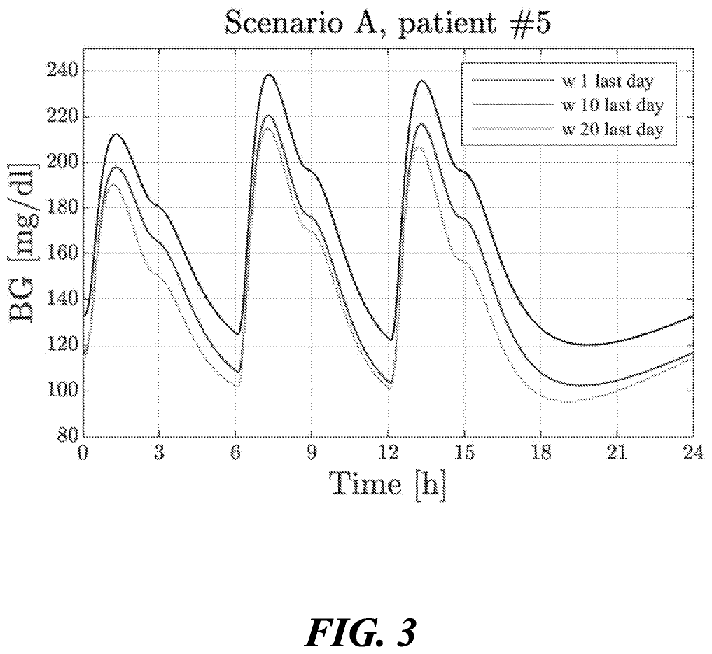

For each patient, the weekly minimum, maximum and mean values [mg/dl] of the drawn SMBG samples were computed, the percent time in range based on a blind CGM, the once-a-day long-acting insulin u₂ doses [U] and average total daily rapid-acting insulin u₁ [U] injected. Population statistics, namely, mean and standard deviation, of the mentioned metrics in Week 1, Week 10 and Week 20 are listed in the columns, and are grouped by scenarios in each set of rows. Significance of the results between Week 1 and Week 10, Week 1 and Week 20 and Week 10 and Week 20, respectively, was evaluated with paired t-tests at the 5% significance level and is listed in the last three columns. It is noted that Week 1 followed conventional therapy and serves as a reference when assessing the performances of the DSS in Week 10, which highlights the results relative to the last ILC iteration, and Week 20, which reports performances relative to the last run of the R2R algorithms, respectively. Conventional therapy consisted in Gla-100 doses of 0.4 U/kg of body weight and nominal carbohydrate-ratios, and was maladjusted to varying extents, e.g., treatment parameters were excessive or insufficient for some in-silico patients, but also close to optimal for some other. That said, in all the scenarios, the effect of the ILC is to decrease basal blood glucose concentration and reduce glucose variability across the population bringing the population average blood glucose measured by SMBG in Week 10 to 136.5(16.1) [mg/dl] in the nominal case, 136.4(15.9) [mg/dl] for perturbed meal sizes and finally 130.3(12.4) [mg/dl] when, in addition to the perturbed meal sizes, SMBG samples are drawn with some anticipation or delay with respect to the protocol. The reduction in glycemic variability can also be seen in the smaller variance for population average minimum, maximum and mean blood glucose measured by SMBG. Further, percent time in range (70,180) [mg/dl] is statistically significantly improved (66.8(33.8)% vs. 90.5(10.5)%, p=0.05 in Scenario A, 66.9(33.1)% vs. 90.3(10.5)%, p=0.05 in Scenario B and 67.2(32.8)% vs. 92.3(9.7)%, p=0.03 in Scenario C) and times spent in severe hypoglycemia as well as hyperglycemia are reduced (percent time <54 [mg/dl] 9.7(23.6)% vs. 0.0(0.0)% in Scenario A, 9.9(23.9)% vs. 0.3(1.0)% in Scenario B, 10.1(24.2)% vs. 0.7(2.3)% in Scenario C; percent time >250 [mg/dl] 2.8(8.8)% vs. 0.0(0.0)% in Scenario A, 2.9(9.1)% vs. 0.0(0.0)% in Scenario B and C. Similar considerations apply to the R2R controller performances. The overall effect is to further reduce glycemic variability seen in Week 20 across the population, while at the same time trying to meet the glycemic targets of 100 and 160 [mg/dl], respectively, for the minimum and maximum BG values. Percent time in range (70,180) [mg/dl] further increases (94.6(6.8)%, p=0.03 in Scenario A, 93.6(6.7)%, p=0.02 in Scenario B and 93.8(7.0)%, p=0.02 in Scenario C). As pointed out earlier, the glycemic outcomes are not affected by variations in meal disturbances. As expected, the timing of the SMBG samples is critical especially for the ILC algorithm which relies upon fasting blood glucose samples. This is evident in the larger amount of u2 delivered in Scenario C, from 28.3(5.0) [U] in Week 1 to 34.3(7.9) [U] in Week 20. Hypoglycemic events, i.e., a measured blood glucose level below 70 [mg/dl], were not reported by self-monitoring of blood glucose in any of the scenarios. FIG. 3 allows a visual evaluation of the improvements introduced with the ILC and R2R controller, respectively, by showing the blind CGM profile of representative patient #5 on Scenario A, for the last day of Week 1, Week 10 and Week 20.

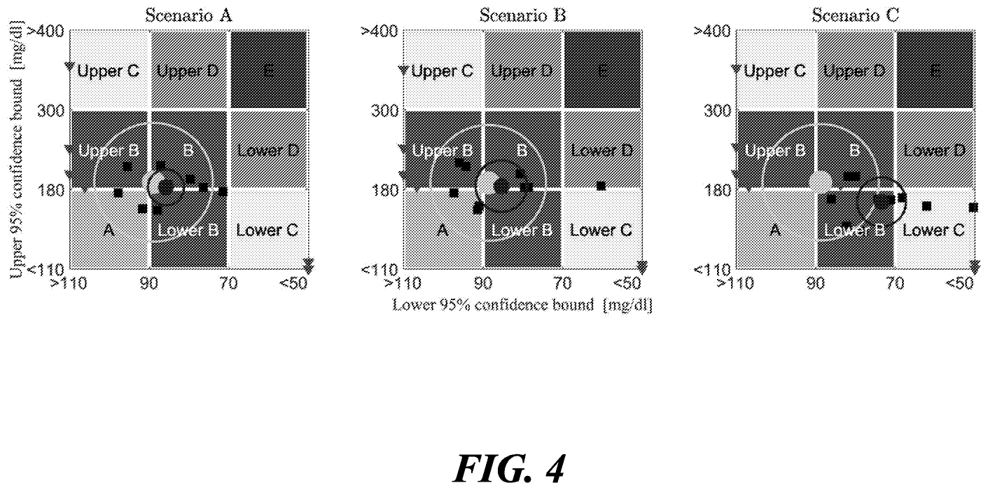

The Control-Variability Grid Analysis [31] was used to assess performances of the proposed strategy using the blind CGM and is given in FIG. 4. The comparison is performed between Week 1 and Week 20. In Scenario A and B the effect of the DSS is to cluster the points around the middle of the green zone, in the bottom left of the grid. Further, whereas the mean of the points has experienced a minor shift in Week 20 compared to Week 1, the standard deviation (circle radii) is much smaller in Week 20 than in Week 1, with the performances in Scenario A being the best, as expected. Control performances in Scenario A and B are similar, with the exception of one hypoglycemic episode in Scenario B (point at the boundary between Lower C and Lower D zone), showing robustness against variation in meal disturbances. Scenario C shows points relative to Week 20 in the lower C zone and exhibits a larger shift in mean and biggest standard deviation from open-loop to closed-loop. This highlights once more the sensitivity of the proposed learning algorithms to the timing of the blood glucose samples used for feedback, in particular for basal doses.

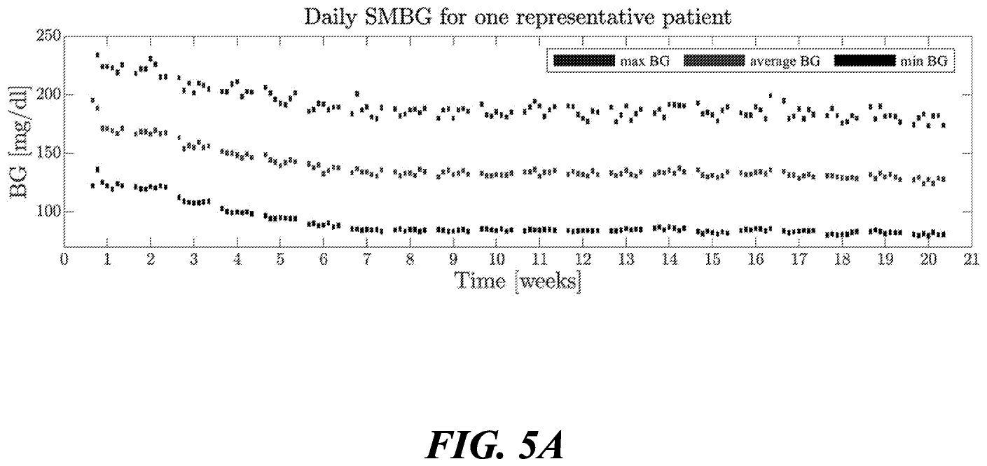

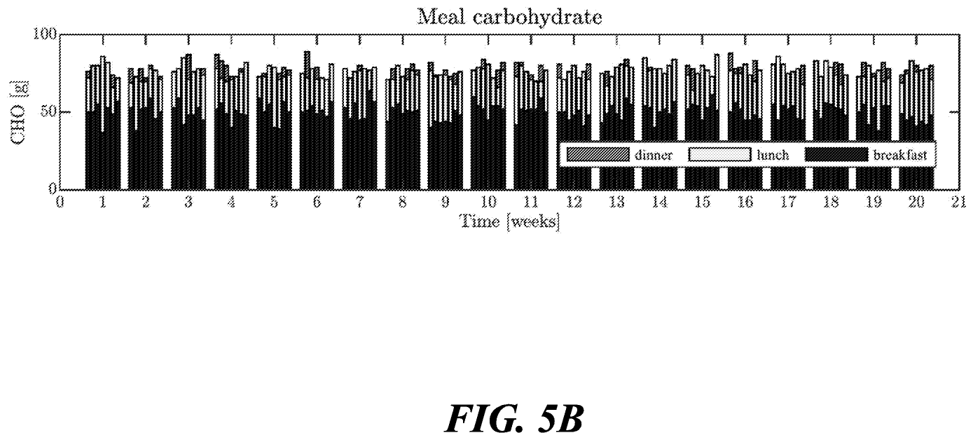

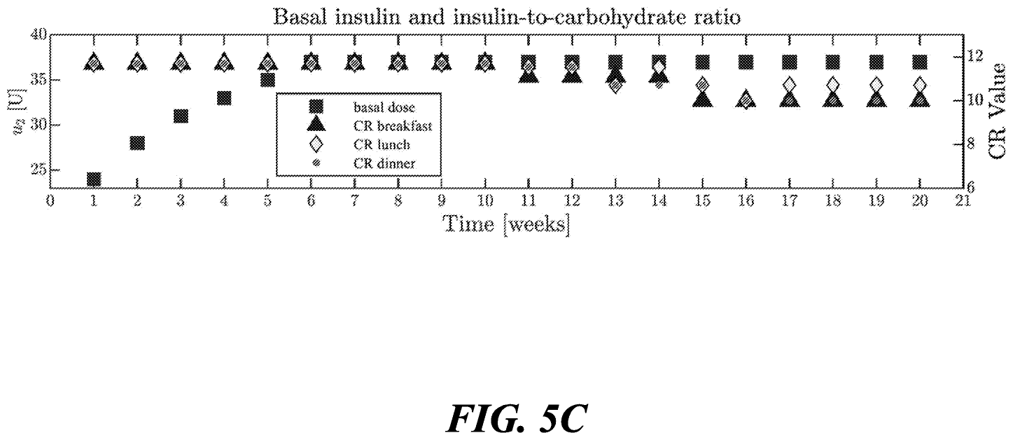

Finally, an illustration of the overall MDI therapy optimization procedure is given for Scenario B, in-silico patient #5, in FIG. 5. The daily maximum, average and minimum SMBG, grouped by simulation week, are portrayed along with the inputs. From a starting u_{2,0}=24 [U] the ILC algorithm attains convergence at iteration #6, the optimal recommended dose is u₂*=37 [U] and is kept constant throughout the remainder of the simulation. The effect of this increased dosage is to bring the average minimum blood glucose to 107.5 [mg/dl], close to the reference 110 [mg/dl]. The R2R algorithm commences on Week 11 and brings blood glucose further down. Note that for this in-silico patient, convergence is achieved for all three meals. Tighter glucose control can be achieved by setting tighter references. However, due the extremely sparse nature of the feedback signal, it was decided to prioritize safety.

### 7. Summary and Conclusion

A novel insulin therapy optimization strategy suitable for patients following the MDI treatment regime in conjunction with SMBG was proposed and tested in simulations using a metabolic model of T1D able to simulate basal and prandial insulin injections. Purpose of the metabolic model was to provide a testing platform, tuned to reflect clinical data from MDI users, able to reproduce the MDI treatment. The development of a validated T1D simulator for MDI therapy was extraneous to this working example, since the idea of learning controllers proposed herein can be applied to any given model of MDI treatment.

In all the tested simulations, initial conditions for all the insulin states were set to zero. Although unrealistic, this choice was twofold: first, it was motivated by the intention of robustifying this approach by deliberately not starting the simulations in steady-state; second, during Week 0 insulin in the various compartments stacked up, so that simulation Week 1 commenced with non-zero initial conditions. The initial conditions derived in this manner are very challenging for these methods and were chosen to show its strengths.

Upon termination of the in-silico trial population mean and standard deviation for the weekly mean blood glucose assessed by SMBGs were 132.5 (12.0) [mg/dl] for the nominal case, 133.2 (10.6) [mg/dl] for the variation in meal sizes case and 125.7 (11.2) [mg/dl] for variation in meal sizes and non-optimal timing of SMBG measurements. Total (basal+bolus) daily insulin was 51.5 (14.2), 51.6 (13.9) and 54.0 (17.2) [U], respectively. Using a blind CGM for monitoring gave time in the target range [70,180] [mg/dl] during Week 20 equal to 94.6%, p=0.03 for Scenario A, 93.6%, p=0.02 for Scenario B and 93.8%, p=0.02 for Scenario C.

Convergence of the ILC algorithm was shown both theoretically and by simulation, while convergence of the R2R algorithm was only shown in simulation. Controller parameters were a function of the body weight and total daily dose and were tuned empirically on the population at hand. Extensive in-silico studies demonstrated that the proposed method proved to be robust against random variations in amount of ingested carbohydrates and, to some extent, against persistent deviations from the protocol when it comes to timing of blood glucose finger-sticks.

### Example 2: Appendix

### A. Subcutaneous Insulin Kinetics

With reference to FIG. 1, the model equations describing subcutaneous insulin kinetics for rapid-acting insulin used in this paper are as follows [21], [22]:

\dot{x}₁(t)=u₁δ(t)−(k_{d}+kₐ₁)x₁(t) (16)

\dot{x}₂(t)=k_{d}x₁(t)−kₐ₂x₂(t) (17)

where u₁ [U] denotes the amount of the injected rapid-acting insulin, x₁ [pmol/kg] the amount of non-monomeric rapid-acting insulin, x₂ [pmol/kg] the amount of monomeric rapid-acting insulin in the subcutaneous space, k_{d} [min⁻¹] the rate constant of insulin dissociation; kₐ₁ [min⁻¹] the rate constant of non-monomeric insulin absorption in plasma; kₐ₂ [min⁻¹] rate constants of monomeric insulin absorption in plasma.

The subcutaneous insulin kinetics of Gla-100 were introduced with a two-compartment model [23], [24]:

\dot{x}₃(t)=−kₛₚx₃(t)+αFu₂δ(t) (18)

\dot{x}₄(t)=−kₐx₄(t)+kₛₚx₃(t)+(1−k)Fu₂δ(t) (19)

where u₂ [mU/kg] is the Gla-100 dose, x₃ and x₄ [mU/kg] the masses of insulin in precipitate and soluble state, respectively, F [dimensionless] the insulin bioavailability, α [dimension-less] the precipitate fraction of the injected dose, kₛₚ [min−1] the rate of dissolution from precipitate to soluble state, and kₐ [min−1] the rate of insulin absorption to plasma.

The rates of appearance of insulin in plasma after an injection of rapid-acting, sᵤ1, and long-acting, sᵤ2, insulin, respectively are thus:

sᵤ1(t)=kₐ₁x₁(t)+kₐ₂x₂(t) (20)

sᵤ2(t)=kₐx₄(t) (21)

### B. Plasma Insulin Kinetics

Plasma insulin kinetic is described by [21]:

x\dot{ }₅(t)=sᵤ1(t)−(m₂+m₄)x₅(t)+m₁x₆(t) (22)

x\dot{ }₆(t)=−(m₁+m₃)x₆(t)+m₂x₅(t) (23)

x\dot{ }₇(t)=sᵤ2(t)−(m₆+m₈)x₇(t)+m₅x₈(t) (24)

x\dot{ }₈(t)=−(m₅+m₇)x₈(t)+m₆x₇(t) (25)

where x₅, x₇, and x₆, x₈ [pmol/kg] are rapid and long acting insulin, respectively, in plasma and in the liver and m₁, m₂, m₃, m₄, m₅, m₆, m₇, m₈ [min⁻¹] are rate parameters.

### C. Parameter Values

Starting from median, 25^{th} and 75^{th} percentiles of the distributions for k, F, kₛₚ, kₐ, m₅, CLu₂ reported in [23], new distributions were fitted to the reported quartiles. Prior information on the shape of the distributions was incorporated with the aim of recreating as closely as possible the original distributions. Parameter values were, then, randomly drawn from the newly estimated distributions and randomly assigned to each in-silico subject. The rate parameters m₆, m₇, m₈ were then derived according to [21], [23]:

$$\begin{matrix}{m}_{6} = \frac{3}{5} \cdot \frac{C {L}_{{u}_{2}}}{0.6 \cdot BW \cdot {V}_{1}} & ( 26 ) \\ {m}_{7} = \frac{0.6 \cdot {m}_{5}}{0.4} & ( 27 ) \\ {m}_{8} = \frac{2}{5} \cdot \frac{C {L}_{{u}_{2}}}{BW \cdot {V}_{1}} & ( 28 )\end{matrix}$$

An 8-day policy of once-daily subcutaneous administration of Gla-100 with the model were simulated, where each simulated injection amounted to 0.4 U/kg of body weight, and compared the obtained insulin peak values and peak times with those reported in [24], [33]. In the simulations, the peak of 19.26±2.80 [μU/mL] was reached at 290±137.76 [min], wheras in comparison, in [24], [33], the insulin peak of 22.8±6.0 [μU/mL] was attained at 312 μU/mL 186 [min].

### D. Robustness of ILC to Uncertainties Related to Body Weight

Considering the 30% variation in Δk, i.e.,

$$P \pm Δ P = \frac{k \pm Δ k}{375 s + 1} .$$

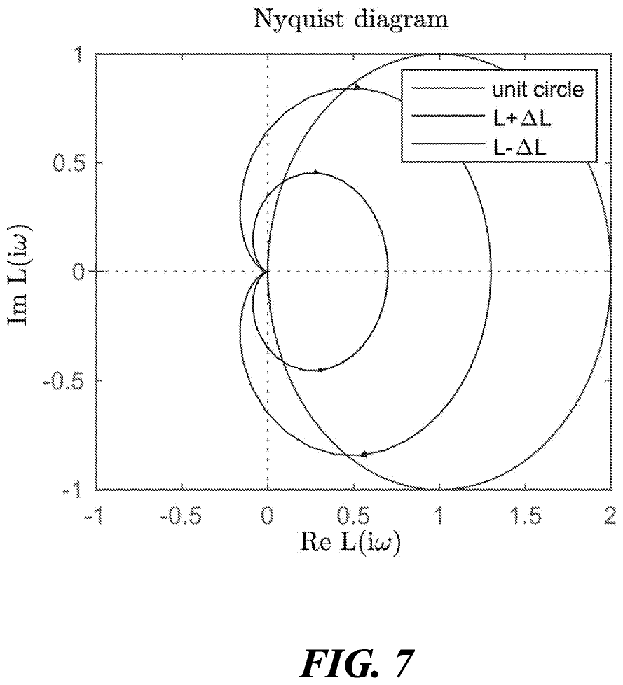

From (7), the Nyquist diagram of L(e^{iw})±ΔL(e^{iw})=[P(e^{iw})±ΔP(e^{iw})]Q(e^{iw})/\hat{P}⁻¹(e^{iw}) is required to stay inside a circle of unitary radius centered in 1. By inspection of FIG. 7 overestimating κ leads to a slower convergence (smaller diagram) but larger margins for robustness (fewer frequency points outside the unit circle) compared to underestimating K.

### E. References

[1] Americ. Diabetes Assoc., “Pharmacologic approaches to glycemic treatment: Standards of medical care in diabetes-2018,” Diabetes Care, vol. 41, no. Supplement 1, pp. S73-S85, 2018.
“Special issue on artificial pancreas systems,” IEEE Control Syst. Mag., February 2018, vol. 38, No. 1.
[3] F. J. Doyle III, L. M. Huyett, J. B. Lee, H. C. Zisser, and E. Dassau, “Closed-loop artificial pancreas systems: engineering the algorithms,” Diabetes Care, vol. 37, no. 5, 2014.
[4] H. Thabit and R. Hovorka, “Coming of age: the artificial pancreas for type 1 diabetes,” Diabetologia, vol. 59, no. 9, pp. 1795-1805, 2016.
[5] A. Haidar, “The artificial pancreas: How closed-loop control is revolu-tionizing diabetes,” IEEE Control Syst. Mag., vol. 36, no. 5, pp. 28-47, 2016.
[6] H. Yeh, T. Brown, N. Maruthur, P. Ranasinghe, Z. Berger, Y. Suh, L. Wilson, E. Haberl, J. Brick, E. Bass, and S. Golden, “Comparative effectiveness and safety of methods of insulin delivery and glucose mon-itoring for diabetes mellitus: A systematic review and meta-analysis,” Ann. Intern. Med., vol. 157, no. 5, pp. 336-347, 09 2012.
[7] D. U. Campos-Delgado, R. Femat, M. Hernandez-Ordonez, and Gordillo-Moscoso, “Self-tuning insulin adjustment algorithm for type 1 diabetic patients based on multi-doses regime,” Appl. Bionics Biomech., vol. 2, no. 2, pp. 61-71, 2005.
[8] D. Campos-Delgado, M. Hernandez-Ordonez, R. Femat, and A. Gordillo-Moscoso, “Fuzzy-based controller for glucose regulation in type-1 diabetic patients by subcutaneous route,” IEEE Trans. Biomed. Eng., vol. 53, no. 11, pp. 2201-2210, 2006.
[9] C. Owens, H. Zisser, L. Jovanovic, B. Srinivasan, D. Bonvin, and F. J. Doyle III, “Run-to-run control of blood glucose concentrations for people with type 1 diabetes mellitus,” IEEE Trans. Biomed. Eng., vol. 53, no. 6, pp. 996-1005, 2006.
[10] F. Campos-Cornejo, D. U. Campos-Delgado, E. Dassau, H. Zisser, L. Jovanovic, and F. J. Doyle III, “Adaptive control algorithm for a rapid and slow acting insulin therapy following run-to-run methodology,” in Proc. Am. Control Conf., 2010, pp. 2009-2014.
[11] Y. Wang, E. Dassau, and F. J. Doyle III, “Closed-loop control of artificial pancreatic β-cell in type 1 diabetes mellitus using model predictive iterative learning control,” IEEE Trans. Biomed. Eng., vol. 57, no. 2, pp. 211-219, 2010.
[12] M. Cescon, S. Deshpande, F. J. Doyle III, and E. Dassau, “Iterative learning control with sparse measurements for long-acting insulin injec-tions in people with type 1 diabetes,” in Proc. Am. Contr. Conf., 2019, pp. 4746-4751.
[13] H. Kirchsteiger, L. Del Re, E. Renard, and M. Mayrhofer, “Robustness properties of optimal insulin bolus administrations for type 1 diabetes,” in Proc. Am. Contr. Conf., 2009, pp. 2284-2289.
[14] M. Cescon, M. Stemmann, and R. Johansson, “Impulsive predictive control of T1DM glycemia: an in-silico study,” in Proc. Dyn. Syst. and Control Conf., 2012, pp. 319-326.
[15] D. S. Carrasco, A. D. Matthews, G. C. Goodwin, R. A. Delgado, and M. Medioli, “Design of MDIs for type 1 diabetes treatment via rolling horizon cardinality-constrained optimisation,” IFAC PapersOn-Line, vol. 50, no. 1, pp. 15 044-15 049, 2017.
[16] K. L. Moore, Iterative Learning Control for Deterministic Systems. London: Springer-Verlag, 1993.
[17] R. W. Longman, “Iterative learning control and repetitive control for engineering practice,” Int. J. Control, vol. 73, no. 10, pp. 930-954, 2000.
[18] M. Norrlo\ddot{ }f, “Iterative learning control: Analysis, design, and experiments,” Thesis no. 653, Linkoping Univ., Linkoping, Sweden, 2000.
[19] D. Bristow, M. Tharayil, and A. Alleyne, “A survey of iterative learning control,” IEEE Control Syst. Mag., vol. 26, no. 3, pp. 96-114, 2006.
[20] Y. Wang, F. Gao, and F. J. Doyle III, “Survey on iterative learning control, repetitive control, and run-to-run control,” J. of Proc. Contr., vol. 19, no. 10, pp. 1589-1600, 2009.
[21] B. P. Kovatchev, M. Breton, C. Dalla Man, and C. Cobelli, “In silico preclinical trials: A proof of concept in closed-loop control of type 1 diabetes,” J. of Diab. Sc. and Tech., vol. 3, no. 1, pp. 44-55, 2009.
[22] B. Kovatchev, M. Breton, C. Cobelli, and C. Dalla Man, “Method, system and computer simulation environment for testing of monitoring and control strategies in diabetes,” 2010, US 2010/0179768 A1.
[23] R. Visentin, M. Schiavon, C. Giegerich, T. Klabunde, C. Dalla Man, and C. Cobelli, “Long-acting insulin in diabetes therapy: In silico clinical trials with the UVA/Padova type 1 diabetes simulator,” in Proc. Int. Conf. Eng. in Med. and Biol. Society, 2018, pp. 4905-4908.
[24] M. Schiavon, R. Visentin, C. Giegerich, T. Klabunde, C. Cobelli, and C. Dalla Man, “Modeling subcutaneous absorption of long-acting insulin glargine in type 1 diabetes,” IEEE Trans. Biomed. Eng., vol. 67, no. 2, pp. 624-631, 2020.
[25] P. Davidson, H. Habblewhite, R. Steed, and B. Bode, “Analysis of guidelines for basal-bolus insulin dosing: basal insulin, correction factor, and carbohydrate-to-insulin ratio,” Endocr Pract., vol. 14, no. 9, pp. 1095-1101, 2008.
[26] J. Walsh and R. Roberts, Pumping Insulin. Torrey Pines Press, 2017.
[27] H. Zisser, L. Jovanovic, F. J. Doyle III, P. Ospina, and C. Owens, “Run-to-run control of meal-related insulin dosing,” Diab. Tech. & Therap., vol. 7, no. 1, pp. 48-57, 2005.
[28] C. Palerm, H. Zisser, L. Jovanovic, and F. J. Doyle III, “A run-to-run framework for prandial insulin dosing: handling real-life uncertainty,” Int. J. of Robust and Nonlin. Contr., vol. 17, pp. 1194-1213, 2007.
[29] F. Campos-Cornejo, D. Campos-Delgado, D. Espinoza-Trejo, H. Zisser, L. Jovanovic, F. J. Doyle III, and E. Dassau, “An advisory protocol for rapid- and slow-acting insulin therapy based on a run-to-run methodology,” Diab. Tech. & Therap., vol. 12, no. 7, pp. 555-565, 2010.
[30] C. Toffanin, R. Visentin, M. Messori, F. D. Palma, L. Magni, and C. Cobelli, “Toward a run-to-run adaptive artificial pancreas: In silico results,” IEEE Trans. Biomed. Eng., vol. 65, no. 3, pp. 479-488, 2018.
[31] L. Magni, D. Raimondo, C. Dalla Man, M. Breton, S. Patek, G. De Nicolao, C. Cobelli, and B. Kovatchev, “Evaluating the efficacy of closed-loop glucose regulation via control-variability grid analysis,” J. of Diab. Sc. and Tech., vol. 2, no. 4, pp. 630-635, 2008.
[32] R. Visentin, M. Schiavon, C. Giegerich, T. Klabunde, C. Dalla Man, and C. Cobelli, “Incorporating long-acting insulin glargine into the UVA/padova type 1 diabetes simulator for in silico testing of MDI therapies,” IEEE Trans. Biomed. Eng., vol. 66, no. 10, pp. 2889-2896, 2019.

### Example 3: Iterative Learning Control with Sparse Measurements for Long-Acting Insulin Injections in People with Type 1 Diabetes

People with type 1 diabetes require exogenous insulin for adequate blood glucose regulation. Traditionally, the clinical therapy consists of multiple daily injections (MDIs) of insulin analogs and a finite number of self-monitoring blood glucose measurements (SMBGs) per day to achieve glycemic regulation. In this working example, the simulation results for once-a-day dosing of long-acting insulin analog using iterative learning control (ILC) to deliver basal insulin are described. To facilitate validation of the control strategy for MDI, modifications to a metabolic model for type 1 diabetes were proposed by adding states related to the subcutaneous insulin kinetics of a generic long-acting insulin. Simulations on the cohort of in-silico patients demonstrate the proposed strategy and its advantages over current clinical practice for basal insulin delivery. In particular, the ILC performs robustly under induced insulin resistance.

### 1. Introduction

Diabetes describes a group of metabolic diseases characterized by high blood glucose levels (hyperglycemia), caused by lack of insulin secretion, impaired insulin action or both. The chronic hyperglycemia has multiple effect throughout the body associated with damage, dysfunction and failure of various organs, especially the eyes, kidneys, nerves, heart and blood vessels [1]. Exogenous insulin replacement is the mainstay of the therapy and it aims at mimicking as closely as possible the physiological insulin secretion pattern in the individual without diabetes consisting in a slow basal secretion throughout the day and an augmented rate at meal times. The majority of people requiring exogenous insulin rely upon multiple daily injections (MDIs) and a finite number of self-monitoring blood glucose measurements (SMBGs) per day [2].

The MDI treatment comprises the delivery of two types of insulin formulations: a long-acting analog (e.g., insulin glargine or insulin degludec) to maintain glucose homeostasis during fasting and a superimposed rapid-acting analog (e.g., insulin lispro or insulin aspart) at meal times to compensate for the glycemic excursions due to the macronutrient content of the ingested food or as a correction for hyperglycemia [3]. The calculation of dose and timing for each type of insulin is left to the individual, following the recommended treatment profile provided by the physician. Generally, the starting insulin doses are based on body weight and are revised by incremental adjustments based on guidelines until a desirable level of control is attained [3].

Several algorithms for automated adjustments of MDI regimes have been proposed in the literature, ranging from PID tuning rules coupled with expert rules [4], fuzzy logic controllers [5], run-to-run (R2R) strategies [6], [7], gain scheduling linear model predictive control (MPC) [8], asymmetric weight line search optimization [9], impulsive predictive control [10] and, more recently, rolling horizon cardinality-constrained optimization [11]. Most of these works focused solely on the meal boluses calculation, assuming the optimal basal therapy, comprised of one or more long-acting insulin doses per day, was given.

Provided herein are methods of iterative learning of long-acting insulin doses was applied to track a basal glucose concentration reference in people with type 1 diabetes. The choice of a learning controller is motivated by the fact that basal insulin therapy is repetitive in nature and people with type 1 diabetes inject long-acting insulin every day under the same conditions, e.g., every morning before breakfast or every night at bedtime. Under these circumstances, the tracking error calculated over an iteration consisting of, e.g., 24 hours, is repeatable and can be used to adjust the long-acting insulin dosing for the next iteration in order to progressively improve the reference tracking, assuming some knowledge about the system describing glucose excursions in response to basal therapy is available. This is distinctly different to standard R2R approaches in the diabetes control field, e.g., [12] and references therein, in which clinically relevant performance metrics such as the rate of change of blood glucose con-centration in the postprandial period or time above/below a predefined target zone measured using data from the past run were used to adapt insulin therapy for the next run mostly based on heuristics.

The main contribution of the paper is the design and in-silico verification of a control policy for basal insulin delivery based on iterative learning control (ILC). These key ideas of a learning controller were applied to a generic model of MDI therapy and tested the proposed strategy on a population of 10 in-silico MDI patients, showing that the algorithm converges using a once-a-day injection policy that produces desired glycemic outcomes under challenging conditions of fasting, meals and meals with induced insulin resistance.

The layout of the remainder of the paper is as follows: in Section 2 a description of the model used for the in-silico patients is provided, in Section 3 the iterative learning controller design is outlined, simulation results obtained using the proposed strategy on the simulated patients are presented in Sec. 4 and finally in Section 5 is a discussion on the findings and conclusions.

### 2. Metabolic Model

In order to facilitate the synthesis of control algorithms for MDI treatment and to verify its feasibility and effectiveness on a population of virtual patients prior to their deployment in clinical studies, a mathematical model of the glucose-insulin metabolic system in people requiring insulin was developed according to a generic MDI therapy. The proposed model is an extension to the meal simulation model of the glucose-insulin system first proposed by Dalla Man and co-workers in [13], [14]. Briefly, the metabolic model is comprised of a gastro-intestinal subsystem which describes digestion and absorption of the carbohydrate content of a meal and subsequent rate of appearance of glucose in blood; a glucose subsystem describing insulin-independent utilization in plasma and fast-equilibrating tissue, and insulin-dependent utilization in the periphery; endogenous glucose production, SC insulin delivery and absorption subsystem for rapid-acting insulin analog; and finally an insulin subsystem representing the absorbed insulin in liver and plasma. For more details, see, e.g., [13], [14].

### A. Insulin Types and Modeling Approach

Once injected into the SC tissue, insulin undergoes trans-formation and transport into the systemic circulation over time. Modern insulin analogs are engineered to vary in the rate of this absorption, among other approaches, to achieve different glycemic goals. A primary mechanism is to use fractions of available injected insulin present as monomers and non-monomers [15], [16]. For rapid absorption profile, as desired after a meal, the fraction of monomers is higher in rapid-acting insulin formulations whereas it is lower for long-acting insulin formulations. Thus, the absorption dynamics acts as a rate determining step in the overall insulin dynamics [16]. The insulin formulations can be differentiated by their half-life which varies from 2 hours for rapid-acting insulin to more than 12.5 hours for long-acting insulin [16], [17]. In the case at hand, it was assumed that the blood glucose concentration after an injection of long-acting insulin reaches its dip at about 12.5 [h] with inflection points roughly at 9 and 26 [h], respectively.

Schiavon and co-workers recently reviewed models of fast-acting insulin absorption from the subcutaneous depot [18] while Visentin et al. developed a pharmacokinetic model of insulin glargine 100 U/mL and incorporated it into the UVa/Padova metabolic simulator [19]. SC absorption and the appearance in plasma of long-acting insulin were observed along with the rapid-acting. In the following example it was contemplated that different insulin formulations have different absorption behaviors, but once insulin reaches plasma, the distribution and elimination are the same.

### B. Subcutaneous Insulin Kinetics

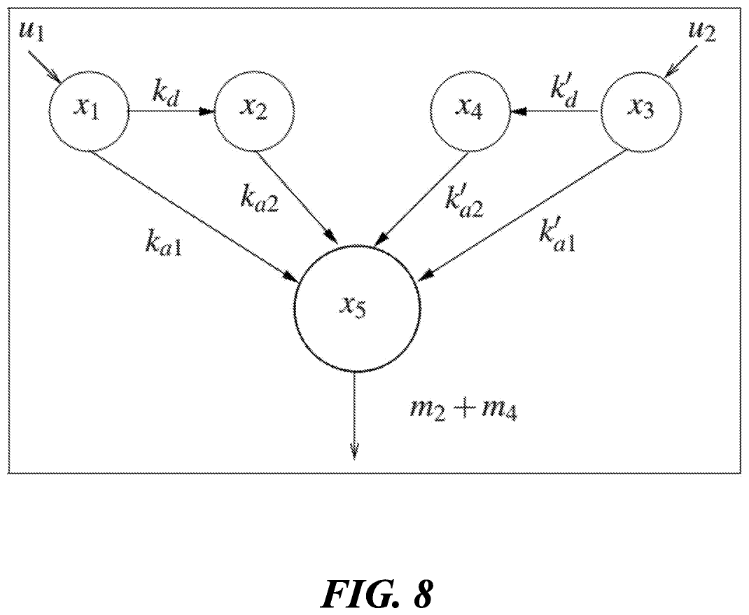

In this work, the SC insulin kinetics model described in [14], [18] was modified as depicted in FIG. 8. Denoting the amounts of the injected rapid-acting and long-acting insulin by u₁ and u₂, respectively, the model equations are as follows:

\dot{x}₁₁(t)=u₁·δ(t)−(k_{d}+kₐ₁)·x₁(t),x₁(0)=0 (1)

\dot{x}₁₂(t)=k_{d}·x₁(t)−kₐ₂·x₂(t),x₂(0)=0 (2)

\dot{x}₁₃(t)=u₂·δ(t)−(kd′+ka′1)·x₃(t),x₃(0)=0 (3)

\dot{x}₁₄(t)=kd′·x₃(t)−ka′2·x₄(t),x₄(0)=0 (4)

where x₁ and x₃ [pmol/kg] are the amount of non-monomeric rapid-acting and long-acting, respectively insulin, x₂ and x₄ [pmol/kg] are the amount of monomeric rapid-acting and long-acting, respectively, insulin in the subcutaneous space, k_{d} and kd′ [min−1] are rate constants of insulin dissociation; kₐ₁ and ka′1 [min−1] are rate constants of non-monomeric insulin absorption in plasma; kₐ₂ and ka′2 [min−1] are rate constants of monomeric insulin absorption in plasma and δ is the delta function. The rate of appearance of insulin in plasma after an injection is thus:

s_{c}(t)=kₐ₁·x₁(t)+kₐ₂·x₂(t)+ka′1·x₃(t)+ka′2·x₄(t) (5)

Finally, insulin in plasma and its degradation was modeled in the liver and periphery as a chain of 2 compartments:

\dot{x}₁₅(t)=s_{c}(t)−(m₂+m₄)·x₅(t)+m₁·x₆(t),x₅(0)=0 (6)

\dot{x}₁₆(t)=−(m₁+m₃)·x₆(t)+m₂·x₅(t),x₆(0)=0 (7)

I(t)=x₅(t)/V_{I} (8)

where x₅ and x₆ [pmol/kg] are insulin in plasma and in the liver, respectively, I [pmol/l] is plasma insulin concentration, V_{I} is the distribution volume of insulin and m₁, m₂, m₃, m₄[min−1] are the parameters as in [13].

### C. Long-Acting Absorption Parameters

In order to mimic the half-life of long-acting insulin, the parameters of rapid-acting insulin absorption were scaled by a factor λ. For simplicity, all three parameters were scaled by the same factor. Thus, kd′=k_{d}/λ is the rate constant between two compartments, ka′1=kₐ₁/λ is the rate constant of first compartment absorption in plasma and ka′2=kₐ₂/λ is the rate constant of second compartment absorption in plasma (see FIG. 8). A bisection method was used to calculate the desired scaling factor by specifying the half-life variable as 12.5 hours and the error tolerance as 0.05 U to preserve inter-subject variability in absorption profiles. Note that A can be tuned to recreate absorption profiles of other types of long-acting insulin formulations.

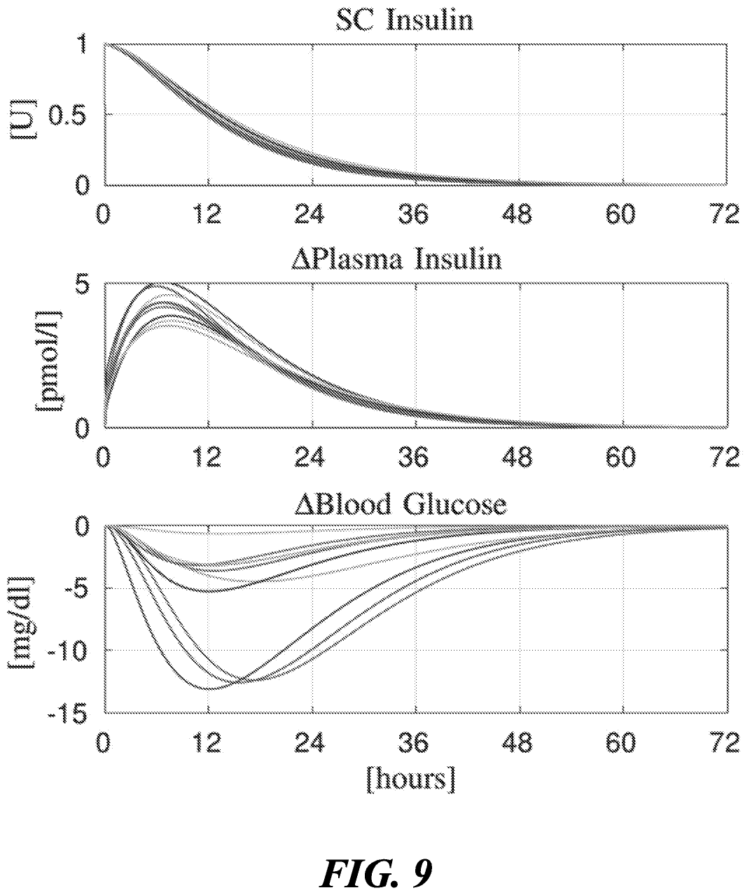

Using the parameters in [20], an in-silico population of 10 people with type 1 diabetes was generated. Table I reports parameters for rapid-acting insulin absorption, the scaling factor λ, patient's body weight and insulin-to-carbohydrate ratio. FIG. 9 shows simulations obtained with the proposed insulin model starting from steady state with u₁=0, u₂=1 [U] for the in-silico population.

### 3. Iterative Learning of Long Acting Insulin

Let the blood glucose excursion due to a bolus of long-acting insulin be described by a discrete-time, linear-time-invariant (LTI), single input-single output (SISO) system:

yⱼ(k)=P(q)·u_{2,j}δ(k) (9)

where y denotes blood glucose concentration, u₂ long-acting insulin, j is the iteration index, k the discrete-time index and q the delay operator. Note that for simplicity it is assumed that no disturbances are acting on the system. The tracking error i.e., the difference between the measured and the desired basal glucose concentration r(k) is defined as:

eⱼ(k)=r(k)−yⱼ(k) (10)

The ILC algorithm developed in this work finds the input u_{2,j}δ(k) to (3) which drives the measured blood glucose concentration as close as possible to the desired reference r(k) by using an iterative method:

u_{2,j+1}δ(k)=u_{2,j}δ(k)+Q(q)L(q)·eⱼ(k) (11)

where Q is a zero-phase low pass filter and L is called learning filter. Note that the filters Q and L can be either causal or non-causal as they operate on signals from the previous iteration of the algorithm. Various methods have been proposed in the literature for the design of L (see, e.g., [21], [22]). In this work, a model-based approach was used, in which the inverse of an approximate model \hat{P}(q) of the actual dynamics P(q) between long-acting insulin injection and blood glucose is used as learning filter.

### A. Approximation P(q) of the Actual Dynamics P(q)

The impulse response between u₂ and y was approximated with gamma distribution functions [9]:

$$y ( t ) = f ( t ; α ; β ) = \frac{{β}^{α}}{Γ ( α )} {t}^{α - 1} {e}^{- β t} , α > 0 , β > 0 \qquad ( 12 )$$

where t denotes time, t>0 and Γ(α) is the gamma function. If peak time and point of inflection times are known, the corresponding shaping parameters α, β can be calculated:

$$\begin{matrix}{t}_{p e a k} = \frac{α - 1}{β} & ( 13 ) \\ {t}_{infl , i} = \frac{α - 1}{β} + \frac{\sqrt{α - 1}}{β} i = 1 , 2 & ( 14 )\end{matrix}$$

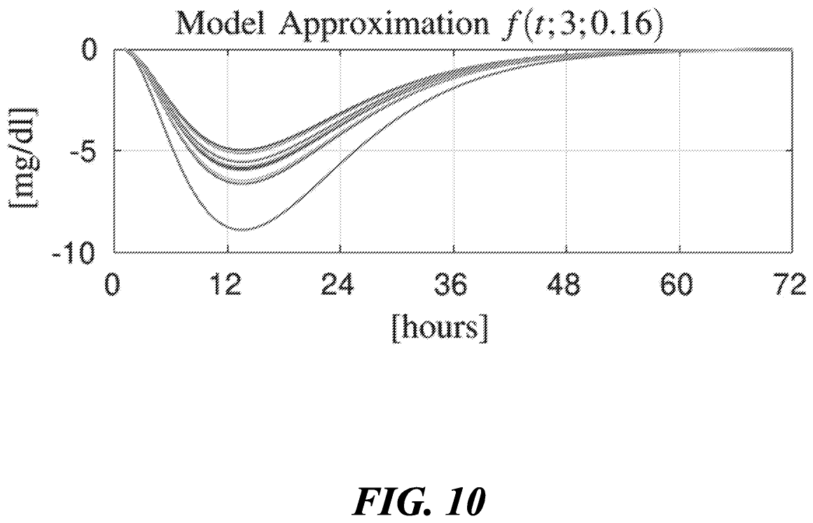

Substituting tₚₑₐₖ=12.5 and t_{in ƒl,1}=9, t_{in ƒl,2}=26, discussed in Sec. 2, in (13) and (14) leads to α=3, β=0.16. Adjusting the gain by a negative scaling factor κ to make the model physiologically plausible and patient specific and taking Laplace transform of (12):

$$P ( s ) = κ \frac{0.004 \; 1}{{( s + 0.16 )}^{3}} \qquad ( 15 )$$

where κ was chosen to represent the patient's body weight reported in Table 4. FIG. 10 depicts the function ƒ(t; 3; 0.16) for the in-silico population considered in this work.

### B. Convergence Properties

It is now of interest to investigate the asymptotic properties of the tracking error. To this end, the following recursive expression is derived:

$$\begin{matrix}{e}_{j + 1} ( k ) = r ( k ) - {y}_{j + 1} ( k ) = r ( k ) - P ( q ) \cdot {u}_{2 , j + 1} ( k ) & ( 16 ) \\ = r ( k ) - P ( q ) ( {u}_{2 , j} ( k ) + Q ( q ) {\hat{P}}^{- 1} ( q ) \cdot {e}_{j} ( k ) ) & ( 17 ) \\ = ( 1 - P ( q ) Q ( q ) {\hat{P}}^{- 1} ( q ) ) \cdot {e}_{j} ( k ) & ( 18 )\end{matrix}$$

TABLE 4
Absorption Parameters For Rapid-Acting Insulin, Scaling
Factor A , Body Weight (BW) And Insulin-To-Carb Ratio (Cr).

| # | k_{d} | kₐ₁ | kₐ₂ | λ | BW (kg) | CR (g/U) |
|---|---|---|---|---|---|---|
| 1 | 0.017 | 0.001 | 0.007 | 4.5625 | 76.4 | 11.6 |
| 2 | 0.017 | 0.002 | 0.016 | 8.1250 | 102.6 | 13.1 |
| 3 | 0.017 | 0.001 | 0.012 | 6.9375 | 74.2 | 7.3 |
| 4 | 0.016 | 0.002 | 0.012 | 6.9375 | 57.3 | 15.7 |
| 5 | 0.015 | 0.002 | 0.020 | 8.1250 | 59.1 | 11.1 |
| 6 | 0.016 | 0.002 | 0.014 | 8.1250 | 68.7 | 10.5 |
| 7 | 0.019 | 0.001 | 0.014 | 8.1250 | 67.3 | 16.4 |
| 8 | 0.013 | 0.001 | 0.012 | 5.7500 | 68.3 | 13.4 |
| 9 | 0.017 | 0.002 | 0.016 | 8.1250 | 64.0 | 8.6 |
| 10 | 0.014 | 0.002 | 0.026 | 10.5000 | 66.6 | 10.9 |

From (18), it can be seen that convergence is achieved if |1−P(e^{iωt}^{ₛ})Q(e^{iωt}^{ₛ})\hat{P}⁻¹(e^{iωt}^{ₛ})|<1

where ω [π, π] and tₛ is the sampling time. In other words, the Nyquist diagram of P(e^{iωt}^{ₛ})Q(e^{iωt}^{ₛ})\hat{P}⁻¹(e^{iωt}^{ₛ}) must be inside a circle of unitary radius centered in 1. The choice of Q affects the robustness of the algorithm and, in turn, is dictated by how far the model \hat{P} is from the true P. In this work,

$$Q ( s ) = \frac{1}{{( s + 1 )}^{2}}$$

was chosen.

### 4. Performance Evaluation

The performances of the proposed controller in simulation were tested on the 10 in-silico MDI patients with type 1 diabetes following MDI therapy previously described in Sec. 2. Specifically, a 20 days protocol was considered starting from 7:00 on Day 0. Long-acting insulin was injected every morning at 7:00 before breakfast. The first 2 days were in open-loop and the corresponding long-acting insulin doses were 0.25 U per kg of body weight, i.e., u_{2,0}=u_{2,1}=0.25 BW. On Day 2, the ILC controller was switched on, providing the first control input on Day 2. Initial conditions for each of the simulations were y(0)=180 [mg/dl] and no insulin in plasma, nor in the subcutaneous depot. Only three SMBG measurements were taken each day at 7:00, 13:00 and 19:00, respectively, and were used by the controller to compute u_{2, j}. The reference basal glucose concentration was r(k)=125 [mg/dl]. Performances of the ILC were evaluated against conventional therapy comprised of one long-acting insulin injection per day of 0.25 U per kg of body weight. Two different scenarios were considered: fasting and three meals a day. Further, the robustness against variations of insulin sensitivity was tested on the second scenario.

### A. Scenario 1: Fasting

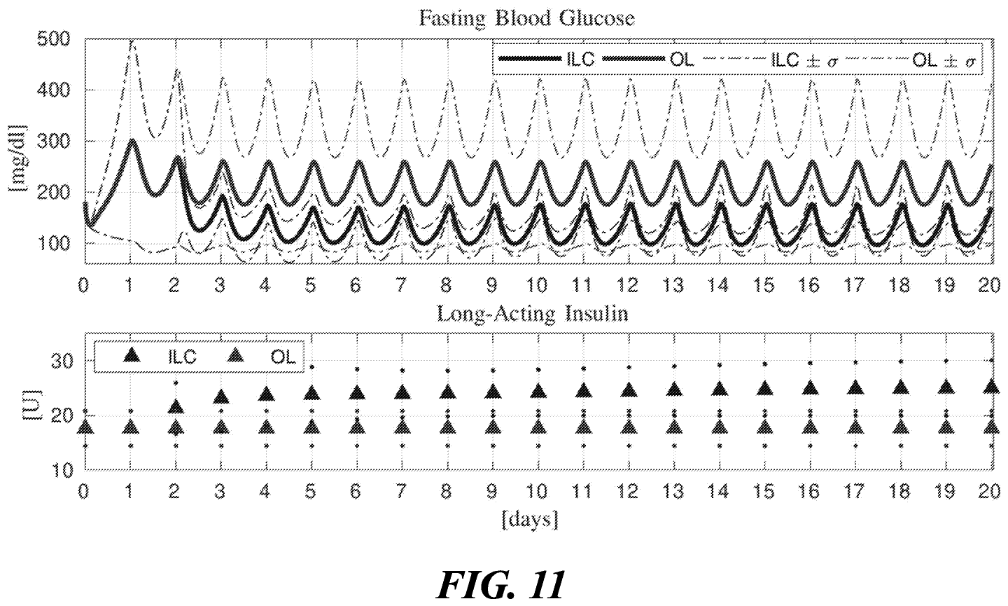

The objective of this simulation was to test whether the controller was able to maintain a basal glucose concentration above the hypoglycemic threshold of 70 [mg/dl] without any intake of meal carbohydrate (CHO) over 20 days. Although unrealistic, this is a particularly challenging situation, as the simulation starts from hyperglycemia with no initial insulin in plasma. As shown in FIG. 11, the mean blood glucose when the ILC controller is turned on is significantly lower than that in open-loop and closer to the desired target. Moreover, there is less variability across patients in the ILC case. The controller is able to converge to the optimal dosing policy within two weeks and insulin doses recommended by the controller are within the plausible range.

### Scenario 2: Three Meals a Day

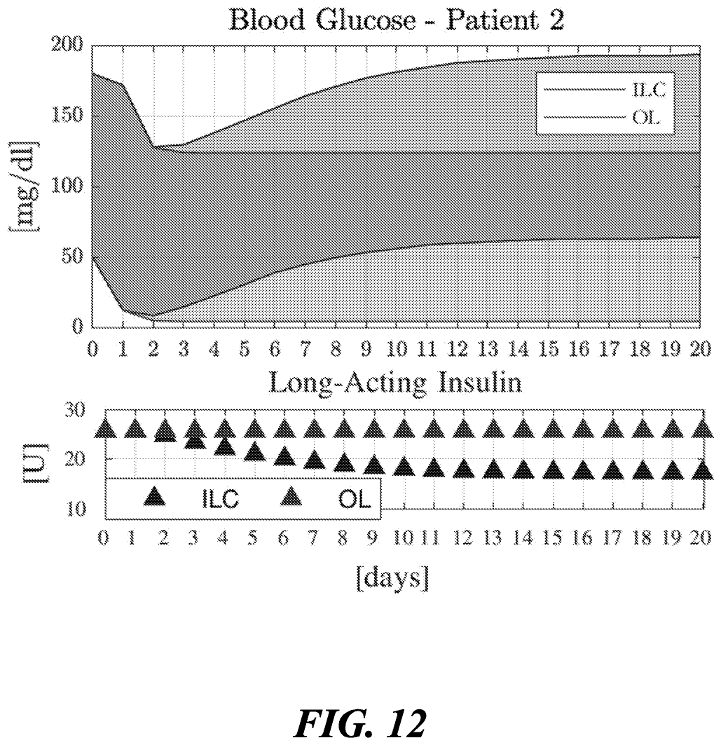

The objective of this simulation was to test the controller in presence of meals and corresponding rapid-acting insulin injections. To this end, on each day, breakfast (50 [g] CHO), lunch (75 [g] CHO) and dinner (75 [g] CHO) were consumed at 7:00, 13:00 and 19:00, respectively. An optimal rapid-acting insulin dose was injected prior to each meal based on the patient specific insulin-to-carbohydrate ratio reported in Table 4. Table 5 reports units of long-acting insulin injected, along with mean blood glucose±standard deviation [mg/dl] on Day 20, per patient. The first and second columns refer to open-loop and ILC case, respectively. From the table it can be seen that the mean blood glucose attained with the controller in the loop was lower than that of the open-loop case. In particular, Patient 2, depicted in FIG. 12: the controller was able to recover from hypoglycemia which had occurred after the first two days in open loop.

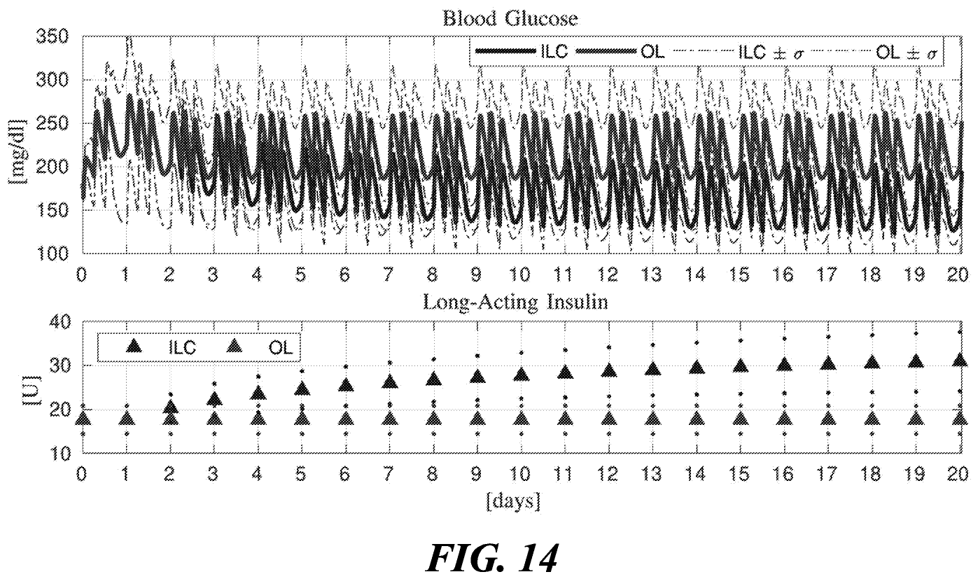

Further, robustness of the proposed algorithm against variations in insulin sensitivity was tested. Insulin resistance was induced by changing the parameters in the metabolic model related to insulin effect on glucose uptake and endogenous glucose production by 40%. As it can be seen in FIG. 14, the controller was able to adapt to the change in insulin sensitivity by delivering more basal insulin, bringing the mean blood glucose closer to the euglycemic range as opposed to the open-loop case. It was noted that meal insulin boluses were not adapted to this case, hence the higher mean blood glucose in the third column of Table 5 compared to the nominal case in the second column.

TABLE 5
PERFORMANCE EVALUATION OF ILC FOR SCENARIO 2.

| | OL | | ILC | | ILC resistant | |
|---|---|---|---|---|---|---|
| # | [U] | [mg/dl] | [U] | [mg/dl] | [U] | [mg/dl] |
| 1 | 19 | 159.22 ± 28.11 | 23 | 145.61 ± 27.38 | 33.7 | 162.60 ± 027.10 |
| 2 | 25.6 | 55.14 ± 34.96 | 17.3 | 121.14 ± 42.98 | 31.1 | 133.33 ± 30.90 |
| 3 | 18.6 | 155.44 ± 39.95 | 16 | 157.14 ± 40.12 | 19.8 | 165.97 ± 41.39 |
| 4 | 14.3 | 293.25 ± 95.67 | 25.3 | 143.16 ± 45.85 | 29.2 | 157.08 ± 24.42 |
| 5 | 14.7 | 193.31 ± 34.87 | 22.6 | 163.69 ± 35.53 | 30 | 179.93 ± 41.23 |
| 6 | 17.1 | 212.60 ± 29.45 | 26.9 | 148.16 ± 30.67 | 43.6 | 164.19 ± 34.18 |
| 7 | 16.8 | 154.51 ± 30.87 | 17.8 | 149.67 ± 30.83 | 22.8 | 160.39 ± 28.32 |
| 8 | 17 | 164.63 ± 51.96 | 20.6 | 124.82 ± 43.93 | 30.1 | 136.11 ± 27.82 |
| 9 | 16 | 191.37 ± 37.94 | 33.8 | 138.66 ± 31.70 | 37.8 | 155.07 ± 19.94 |
| 10 | 16.6 | 169.87 ± 22.63 | 22.2 | 151.85 ± 23.20 | 29.9 | 167.28 ± 28.1 |

### C. Convergence of the Algorithm

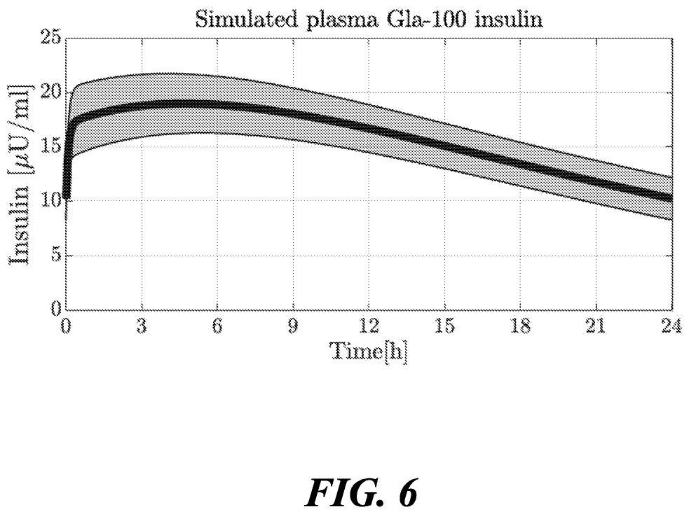

In order to address the transient behavior and the stability properties of the proposed learning controller, the root mean squared of the tracking error (RMSE) was computed as function of the iteration number for each of the scenarios considered in this paper. FIG. 6 shows the population average of such RMSE. The error sharply declines within the first 10 days, with little to no variation in the following 10 days, in 2 out of the 3 cases studied. The case of induced insulin resistance exhibits much slower convergence due to the lack of adaptation of the meal boluses.

### 5. Discussion and Summary

In this work, an extension to the meal simulation model by Dalla Man and co-workers [13], [14] was used to include a long-acting insulin absorption model for the simulation of MDI therapy and proposed an ILC-based once-a-day dosing strategy to provide basal insulin. The simulation results are shown for conditions of fasting, meal and meal with induced insulin resistance. In all three cases, the ILC performs better than the open-loop dose of 0.25 U per kg of body weight, by providing the appropriate amount of basal insulin. In particular, in the case of insulin resistance, it was observed that the ILC delivers more insulin as required while the open-loop does not deliver sufficient insulin. It is worth noting that only three SMBG samples per day taken prior to each meal were used by the controller to compute the dose for the next day. The controller is limited to a decision interval of one day which results in inevitable oscillations at equilibrium, due to the half-life of the long-acting insulin.

A key advantage of the ILC algorithm presented herein is that it does not require an exact representation of the actual dynamics P(q) to successfully track the reference trajectory as opposed to some other model-based control methods. The ILC was initialized with the patient's body weight used in the approximated model and with two days of open-loop therapy and was not privy to any other patient specific parameter like basal rate or insulin sensitivity. It is important to note that the open-loop strategy was chosen to be clinically meaningful, following the guidelines presented in [3], and was the dosing strategy that avoided hypoglycemia in the virtual patients for the fasting simulation. Hence, it was also used as initialization of the ILC.

The learning controller for basal insulin therapy provided herein can be applied to any given model of MDI therapy.

### 6. References

[1] “Classification and diagnosis of diabetes: Standards of medical care in diabetes-2018,” Diabetes Care, vol. 41, no. Supplement 1, pp. S13-S27, 2018.
[2] J. Pickup, “Insulin pumps,” Int. J. Clin. Pract., vol. 65, pp. 16-19, 2011.
[3] “Pharmacologic approaches to glycemic treatment: Standards of medical care in diabetes-2018,” Diabetes Care, vol. 41, no. Supplement 1, pp. S73-S85, 2018.
[4] D. U. Campos-Delgado, R. Femat, M. Hernandez-Ordonez, and A. Gordillo-Moscoso, “Self-tuning insulin adjustment algorithm for type 1 diabetic patients based on multi-doses regime,” Appl. Bionics Biomech., vol. 2, no. 2, pp. 61-71, 2005.
[5] D. Campos-Delgado, M. Hernandez-Ordonez, R. Femat, and A. Gordillo-Moscoso, “Fuzzy-based controller for glucose regulation in type-1 diabetic patients by subcutaneous route,” IEEE Trans. Biomed. Eng., vol. 53, no. 11, pp. 2201-2210, 2006.
[6] C. Owens, H. Zisser, L. Jovanovic, B. Srinivasan, D. Bonvin, and F. J. Doyle III, “Run-to-run control of blood glucose concentrations for people with type 1 diabetes mellitus,” IEEE Trans. Biomed. Eng., vol. 53, no. 6, pp. 996-1005, 2006.
[7] F. Campos-Cornejo, D. U. Campos-Delgado, E. Dassau, H. Zisser, L. Jovanovic, and F. J. Doyle III, “Adaptive control algorithm for a rapid and slow acting insulin therapy following run-to-run methodol-ogy,” in In Proc. Am. Control Conf., 2010, pp. 2009-2014.
[8] H. Kirchsteiger, L. Del Re, E. Renard, and M. Mayrhofer, “Robustness properties of optimal insulin bolus administrations for type 1 diabetes,” in In Proc. Am. Control Conf., 2009, pp. 2284-2289.
[9] H. Trogmann, H. Kirchsteiger, and L. Del Re, “Hybrid control of type 1 diabetes bolus therapy,” in In Proc. Conf. Decis. Control, 2010, pp. 4721-4726.
[10] M. Cescon, M. Stemmann, and R. Johansson, “Impulsive predictive control of T1DM glycemia: an in-silico study,” in In Proc. Dyn. Syst. and Control Conf., 2012, pp. 319-326.
[11] D. S. Carrasco, A. D. Matthews, G. C. Goodwin, R. A. Delgado, and A. M. Medioli, “Design of MDIs for type 1 diabetes treatment via rolling horizon cardinality-constrained optimisation,” IFAC PapersOn-Line, vol. 50, no. 1, pp. 15 044-15 049, 2017.
[12] C. Toffanin, R. Visentin, M. Messori, F. D. Palma, L. Magni, and C. Cobelli, “Toward a run-to-run adaptive artificial pancreas: In silico results,” IEEE Trans. Biomed. Eng., vol. 65, no. 3, pp. 479-488, 2018.
[13] C. Dalla Man, R. A. Rizza, and C. Cobelli, “Meal simulation model of the glucose-insulin system,” IEEE Trans. Biomed. Eng., vol. 54, no. 10, pp. 1740-1749, 2007.
[14] C. Dalla Man, D. Raimondo, R. Rizza, and C. Cobelli, “GIM, simulation software of meal glucose-insulin model,” J. Diabetes Sci. Technol., vol. 1, no. 3, 2007.
[15] A. K. J. Gradel, T. Porsgaard, J. Lykkesfeldt, T. Seested, S. Gram-Nielsen, N. R. Kristensen, and H. H. F. Refsgaard, “Factors affecting the absorption of subcutaneously administered insulin: Effect on variability,” J. of Diabetes Res., vol. 2018, no. 1205121, p. 17, 2018.
[16] T. Heise and L. Meneghini, “Insulin stacking versus therapeutic accumulation: Understanding the differences,” Endocr. Pract., vol. 20, no. 1, pp. 75-83, 2014.
[17] C. Tarin, E. Teufel, J. Pico, J. Bondia, and H. Pfleiderer, “Compre-hensive pharmacokinetic model of insulin glargine and other insulin formulations,” IEEE Trans. Biomed. Eng., vol. 52, no. 12, pp. 1994-2005, 2005.
[18] M. Schiavon, C. Dalla Man, and C. Cobelli, “Modeling subcutaneous absorption of fast-acting insulin in type 1 diabetes,” IEEE Trans. Biomed. Eng., vol. 65, no. 9, pp. 2079-2086, 2018.
[19] R. Visentin, M. Schiavon, C. Giegerich, T. Klabunde, C. Dalla Man, and C. Cobelli, “Long-acting insulin in diabetes therapy: In silico clinical trials with the UVA/Padova type 1 diabetes simulator,” in Proc. Int. Conf. Eng. in Med. and Biol. Society, 2018, pp. 4905-4908.
[20] B. Kovatchev, M. Breton, C. Cobelli, and C. Dalla Man, “Method, system and computer simulation environment for testing of monitoring and control strategies in diabetes,” 2010, US 2010/0179768 A1.
[21] M. Norrlo\ddot{ }f, “Iterative learning control: Analysis, design, and experiments,” Thesis no. 653, Linkoping Univ., Linkoping, Sweden, 2000.
[22] D. Bristow, M. Tharayil, and A. Alleyne, “A survey of iterative learning control,” IEEE Control Syst. Mag., vol. 26, no. 3, pp. 96-114, 2006.

### Example 4: Iterative Learning Control with Sparse Measurements for Long-Acting Insulin Injections in People with Type 1 Diabetes

Objective: Design and in-silico verification of injection policies for long-acting insulin based on iterative learning control (ILC).

A compartment model of subcutaneous insulin absorption was determined (FIG. 8).

Simulation results using the proposed model starting from steady state with u₂=1[U] were performed. Traces show the 10 in-silico patients (FIG. 9).

Automation of Basal Insulin Therapy-ILC for Long-Acting Insulin: Basal insulin therapy is repetitive. Patients inject long-acting insulin every day in a periodic manner. Between days, information about the control quality form the previous day can be learned and used to adjust the long-acting insulin dosing for the next day to progressively improve performances. See, e.g., Zisser et al. JRNC (2007), which is incorporated herein by reference in its entirety.

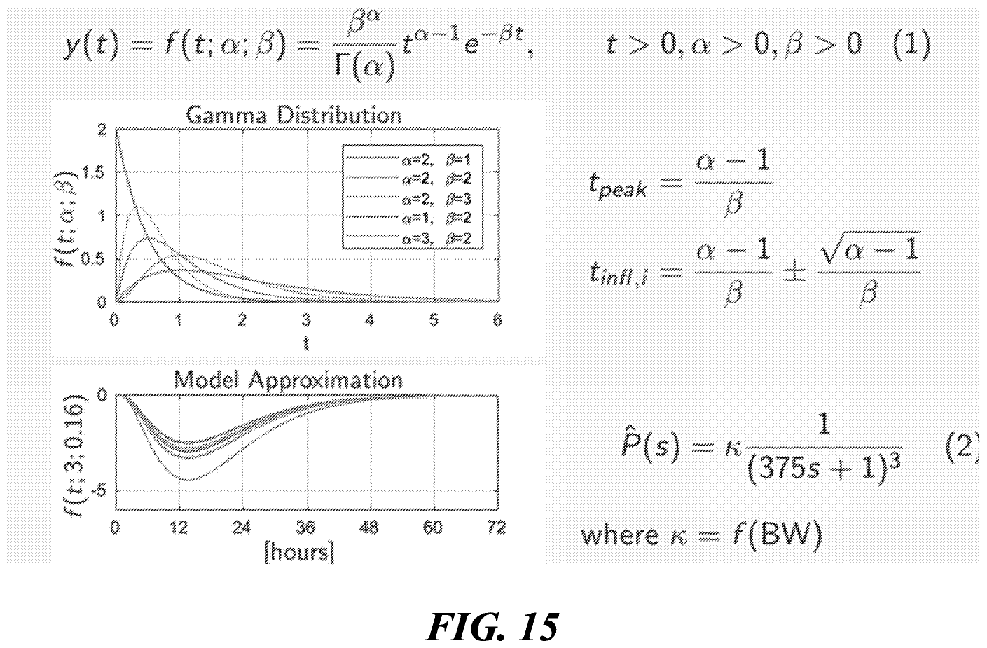

Approximation \hat{P}(q) of the Actual Dynamic P(q). Gamma distribution functions to approximate the impulse response (FIG. 15).

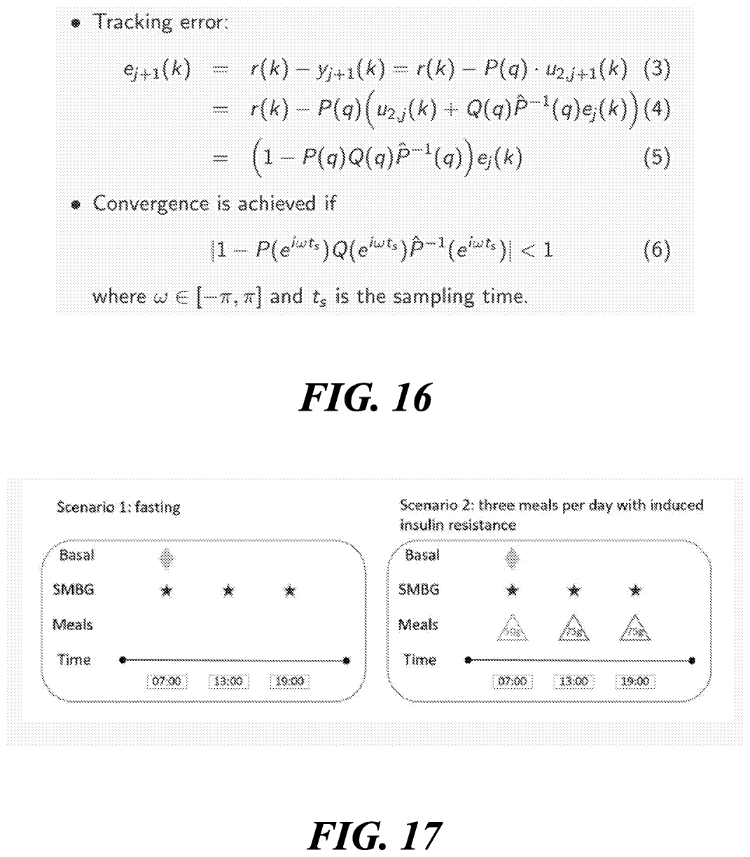

Convergence properties. Tracking error and convergence are shown in FIG. 16. The choice of Q affects the robustness and the speed of convergence of the algorithm.

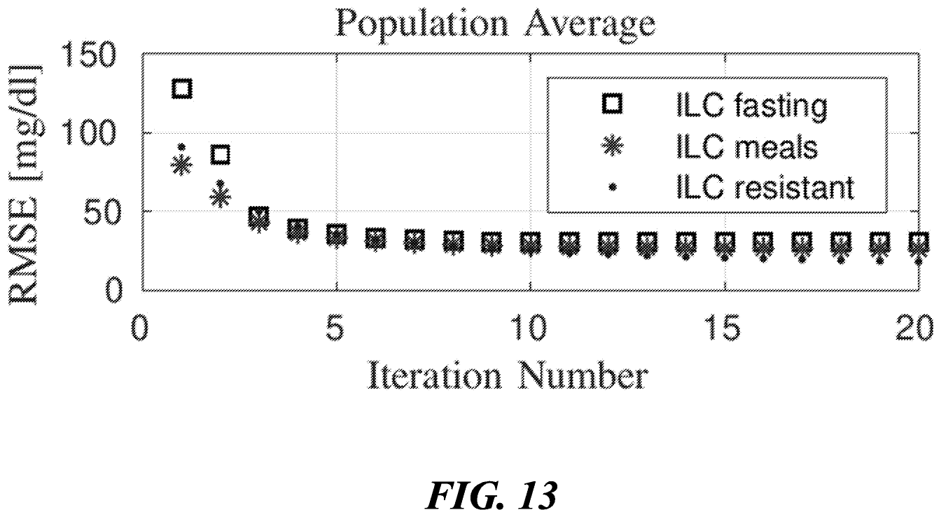

Performance Evaluation. 10 in-silico T1DM patients following MDI therapy were tested using a 20-day protocol, starting from 7:00 on Day 0 (FIG. 17). Performance was evaluated against conventional therapy comprised of one long-acting insulin injection per day of 0.25 U×kg of body weight (BW). See also FIGS. 11-14 and EXAMPLE 2 above.

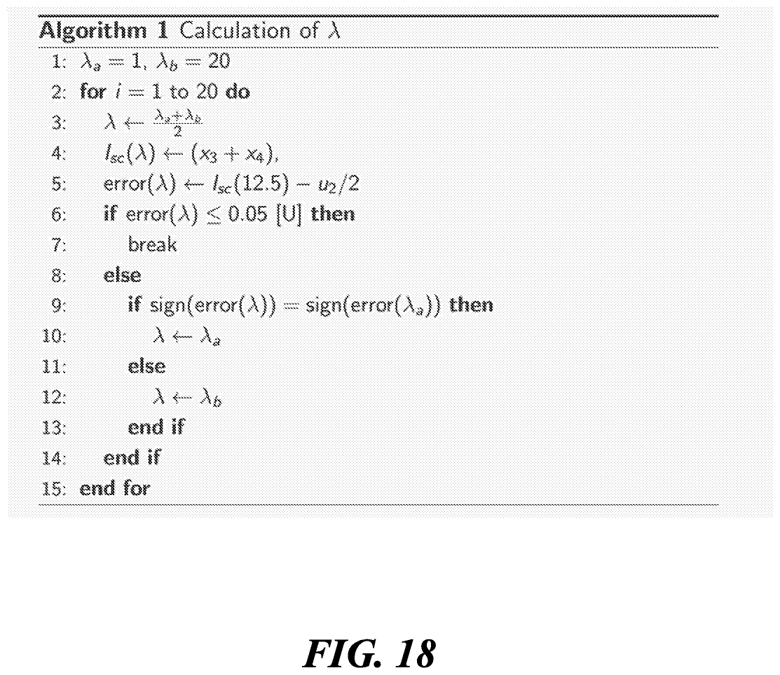

Long Acting Insulin Absorption. The calculation of λ is shown in FIG. 18.

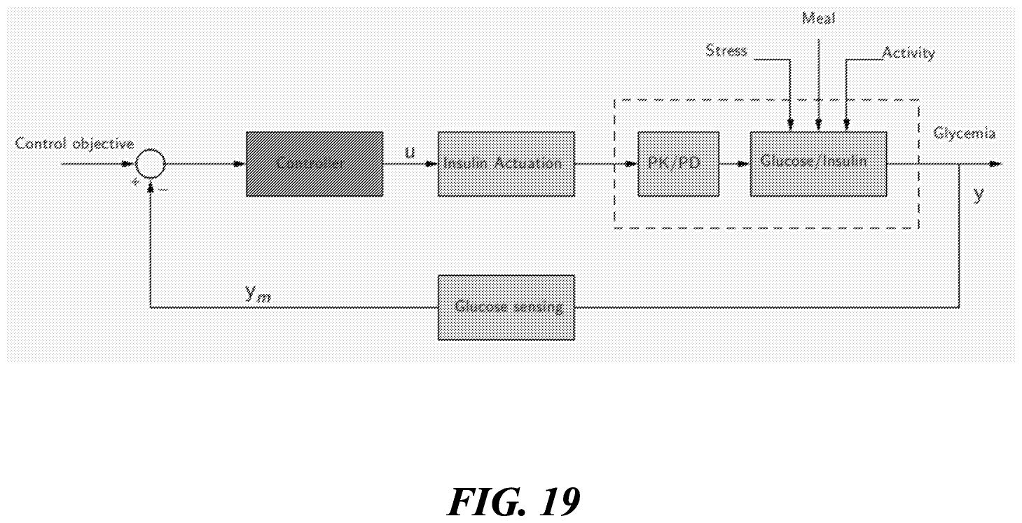

Automated Insulin Delivery and Decision Support Systems. Currently T1D is incurable. However, better treatment outcomes and lifestyle improvements can be attained with automated insulin delivery systems and decision support system. FIG. 19 shows a schematic representation of a support system that exploits dynamics and controls concepts.

Conclusions. Long-acting insulin absorption model tuned to mimic reported half-life of long-acting insulin. ILC-based once-per-day dosing strategy provides basal insulin. The advantages of the methods provide herein are as follows: (1) the controller outperforms traditional therapy; (2) the ILC strategy does not require an exact representation of P(q); (3) the method provided herein only needs 3 SMBG samples per day taken prior to each meal; and (4) the decision interval of one day results in inevitable oscillations at equilibrium due to the drug half-life are considered.

1. A method of updating a basal dose using at least one processor, the method comprising:
determining, using the at least one processor, a tracking error comprising a difference between a measured basal blood glucose concentration and desired basal blood glucose concentration;
determining, using the at least one processor, a basal dose to be administered to a patient based on an Iterative Learning Control (ILC) algorithm wherein using the tracking error; and
storing, in a memory, the basal dose.

2. The method of claim 1, further comprising administering the basal dose to a patient.

3. The method of claim 1, wherein the ILC algorithm is iterated until a desired threshold is reached.

4. The method of claim 3, wherein the desired threshold comprises a convergence.

5. The method of claim 3, wherein the desired threshold comprises a minimization of the tracking error within a threshold window.

6. The method of claim 1, further comprising controlling, using at least one of said at least one processor, the delivery of insulin based on the basal dose.

7. The method of claim 1, wherein the measured basal blood glucose concentration is determined one, two or three times a day.

8. The method of claim 1, wherein the tracking error is determined daily, every two days, or every week.

9. The method of claim 1, wherein the ILC algorithm comprises the formula:
u_{2,j+1}δ(k)=u_{2,j}δ(k)+γQ(q)L(q)ēⱼ(k)
wherein Q represents a zero-phase low pass filter, L is called learning filter, and γ represents a parameter related to a speed of convergence, and ēⱼ(k) is the average error over one iteration of the algorithm.

10. A method of updating a CR value for a Run-to-Run (R2R) controller, using at least one processor, the method comprising:
determining, using the at least one processor, a set of performance parameters based on:
a set of preprandial measured and target glucose values; and
a set of postprandial measured and target glucose values; and
updating, using the at least one processor, a CR profile for a patient preprandial insulin dose to be administered to a patient based on an iterative R2R algorithm using the set of performance parameters; and
storing, in a memory, the updated CR profile.

11. The method of claim 10, further comprising receiving a set of data related to a meal from the patient, determining a preprandial dose based on the set of data and the updated CR profile, and administering the preprandial dose to the patient prior to the meal.

12. The method of claim 11, wherein the patient has type 1 diabetes.

13. The method of claim 11, further comprising controlling, using at least one of said at least one processor, the delivery of insulin based on the preprandial dose.

14. The method of claim 10, wherein the iterative R2R algorithm comprises an update law according to the formula for a set of j iterations:
$$C {R}_{j + 1}^{m} = \{ \begin{matrix}C {R}_{j}^{m} & if & {y}_{j} ( {k}_{pre}^{m} ) > {T}_{b g} ( {k}_{pre}^{m} ) \\ & and & {y}_{j} ( {k}_{p o s t}^{m} ) < {T}_{b g} ( {k}_{p o s t}^{m} ) \\ C {R}_{j}^{m} - ( {c}_{1} {G}_{1} + {c}_{2}^{m} {G}_{2} ) & if & {y}_{j} ( {k}_{p o s t}^{m} ) > {T}_{b g} ( {k}_{p o s t}^{m} )\end{matrix}$$
wherein the subscript m, m∈represents meal type {breakfast, lunch, dinner},
wherein kₚᵣₑ^{m} and kₚₒₛₜ^{m} represent time at which finger-sticks blood glucose samples are drawn,
wherein T_{bg}(kₚᵣₑ^{m}) represent the preprandial glycemic target for each meal m at the time of finger-stick,
wherein T_{bg}(kₚₒₛₜ^{m}) represent the posprandial glycemic target for each meal m at the time of finger-stick,
wherein \tilde{c}₁, [symbol] represent controller gains, wherein c₁=\tilde{c}₁·k̆ and [symbol] = [symbol] ·k̆, and wherein k̆ is a patient-specific constant,
and wherein G₁ and G₂ represent performance metrics determined by the formulae comprising:
$${G}_{1} = \frac{{y}_{j} ( {k}_{pre}^{m} ) - {T}_{bg} ( {k}_{pre}^{m} )}{{T}_{bg} ( {k}_{pre}^{m} )} and {G}_{21} = \frac{{y}_{j} ( {k}_{p o s t}^{m} ) - {T}_{bg} ( {k}_{p o s t}^{m} )}{{T}_{bg} ( {k}_{p o s t}^{m} )}$$

15. The method of claim 1, wherein the set of j iterations continues until measured preprandial and post prandial glucose fluctuations are within a threshold.

16. An artificial pancreas for insulin delivery, the artificial pancreas comprising:
at least one non-transitory memory operable to store program code;
an Iterative Learning Control (ILC) including at least one processor operable to read said program code and operate as instructed by said program code, said program code causing the at least one processor to:
determine a set of performance parameters based on a set of measured and target glucose values on a periodic basis; and
update a set of parameters of the ILC based on the set of performance parameters; and
store, in the at least one non-transitory memory, the set of parameters to output an updated ILC; and
deliver the insulin based on the updated ILC.

17. The artificial pancreas of claim 16, wherein the set of measured and target glucose values comprise basal glucose values.

18. The artificial pancreas of claim 17, wherein the set of measured and target glucose value further comprise preprandial and postprandial glucose values.

19. The artificial pancreas of claim 18, wherein said updating the set of parameters of the ILC comprises updating a CR value.

20. The artificial pancreas of claim 16, wherein said updating the set of parameters of the ILC comprises update a tracking error.

21. The artificial pancreas of claim 16, further comprising a display.

22. The artificial pancreas of claim 21, wherein the display outputs a suggested dose of insulin to a delivery device and/or a subject.
